(19) United States
(12) Patent Application Publication
(10) Pub. No.: US 2011/0055976 A1
(43) Pub. Date: Mar. 3, 2011

(54) Method Of Protease Production In Plants

(75) Inventors: Romy Kandzia, Halle/Saale (DE); Carola Engler, Halle/Saale (DE); Sylvestre Marillonnet, Halle (Saale) (DE); Victor Klimyuk, Halle (Saale) (DE); Yuri Gleba, Berlin (DE)

(73) Assignee: ICON GENETICS GMBH, Munich (DE)

(21) Appl. No.: 12/920,847

(22) PCT Filed: Mar. 3, 2009
(86) PCT No.: PCT/EP2009/001502

§ 371 Date: Oct. 27, 2010

(30) Foreign Application Priority Data
Mar. 4, 2008 (EP) 08004005.8

(51) Int. Cl. C12N 15/87; C12N 9/48; C12N 9/76; C12N 15/63; A01H 5/00; C12N 5/10

(52) U.S. Cl. 800/288; 435/219; 435/468; 435/213; 435/320.1; 800/298; 435/419

(57) ABSTRACT

A process of producing a protease in a plant or in plant cells, comprising (a) providing a plant comprising a heterologous nucleotide sequence comprising a coding sequence encoding a fusion protein, said fusion protein comprising: an apoplast or plastid signal peptide; a SUMO protein or a derivative of a SUMO protein; and a zymogen of said protease, and (b) expressing said fusion protein.

### FIELD OF THE INVENTION

The present invention relates to a process for the production of a protease of interest in a plant or in plant cells and protease obtained thereby. Further, the invention relates to a nucleotide sequence and a vector used for the process of the invention and to a transgenic plant or plant cells containing the nucleotide sequence or vector. Further, the invention relates to the use of a SUMO protein or a derivative thereof for expressing a protease of interest in a plant or in plant cells. vectors for this process and to plants or plant cells transformed therewith.

### BACKGROUND OF THE INVENTION

Recombinant protein production in plant systems has been very successful for many different products, covering proteins with industrial applications, food and feed additives, animal health products and human pharmaceuticals, such as antigens and immune response proteins.

There are many comprehensive reviews describing the field (Hood & Jilka, 1999, Curr. Opin. Biotech., 10:382-386; Doran, 2000, Curr. Opin. Biotech., 11:199-204; Daniell, et al., 2001 Trends Plant Sci., 6:219-226; Larrick & Thomas, 2001, Curr. Opin. Biotech., 12:411-418; Klimyuk et al., 2005, in: Modern Biopharmaceuticals, ed. J. Knaeblein, WILEY-VCH, Weinheim, 893-917; Gleba et al., 2007, Curr Opin. Biotechnol., 18, 134-141). Plants have been considered as a low-cost production system for proteins, that is significantly cheaper in comparison with bacterial, yeast, insect and mammalian cell-based production systems. The available data in this regard confirm the above said.

However, a challenge still exists when a commercially applicable expression level of cytotoxic proteins such as proteases has to be achieved. Proteases are used in many different commercial applications, including pharmaceutical and laboratory uses. The sources of proteases are usually either animal tissues (e.g. bovine and porcine pancreas for trypsin and chymotrypsin production) or bacteria (e.g. strains of Bacillus for subtilisin and thermolysin production). In the case of animal tissue as the source of proteases, potential cross-contamination by undesirable components (e.g. prions or other infectious agents) produced by the animal cells must be taken care of. Bacterial production of many proteases is rather restricted due to their low yield that in many cases is well below commercially viable levels. Kilogram quantities of proteases are often required for industrial scale applications.

Production of chymotrypsin (US2006015971) and trypsin (US6087558) in plants (corn grain) was previously described. This method provides a plant source of recombinant proteases with several of the advantages of a plant production host. In a prior art plant expression system for trypsin (Woodard et al., 2003, Biotechnol. Appl. Biochem., 38 123-130), trypsin was targeted in the zymogen form to the cell wall in corn seeds using an embryo-preferred promoter in transgenic maize plants. Several other promoter or subcellular target sites gave inferior expression yields. Seeds were considered ideal for trypsin expression due to their content of trypsin inhibitor that may minimize detrimental effects of trypsin on the host plant.

However, it follows from the published data (Woodard et al., 2003, Biotechnol. Appl. Biochem., 38 123-130) that the highest expression level of trypsin obtained in corn seeds is rather low (ca. 58 mg/kg of corn grain), which inevitably affects the market cost of recombinant trypsin. Indeed, the cost of plant-derived recombinant trypsin is significantly higher in comparison with that from traditional sources. This is not surprising as the cost of recombinant protein production (including downstream processing) is reversely dependent on the expression level of the protein. For example, extraction of beta-glucuronidase (GUS) from transgenic corn seeds accounts for 94% of the production cost (Evangelista et al., 1998, Biotechnol. Prog., 14:607-614). The calculations were done for transgenic corn containing 0.015% of recombinant GUS. It was stated that increase in recombinant GUS expression level up to 0.08% (4-5 folds) significantly improves the process economics. Also, the production of recombinant protease in agriculturally important plant that is commonly used in feed/food chains creates additional biosafety risks of cross-contaminating non-transgenic seed stock.

Departing from the prior art, it is the problem of the invention to provide a plant expression system and production process for proteases that gives high yield and allows industrial scale quantities of the protease to be produced. It is a further object of the invention to provide a plant expression system for proteases that avoids contamination of food, feed or seed stock intended for human or animal consumption by transgenic plant material. It is a further object of the invention to provide a process of producing a protease of interest in plants or plant cells, which addresses the problems associated with toxicity of said protease to plant cells expressing the protease and offers an economic way of the protease production in plants.

### GENERAL DESCRIPTION OF THE INVENTION

The present invention provides a process of producing a protease in a plant or in plant cells, comprising
(i) providing a plant comprising a nucleotide sequence comprising a coding sequence encoding a fusion protein, said fusion protein comprising an apoplast or plastid signal peptide, a SUMO protein or derivative of a SUMO protein, and a zymogen of said protease, and
(ii) expressing said fusion protein.

The present invention further provides a process of producing a protease in a plant or in plant cells, comprising providing a plant with a replicon comprising a coding sequence encoding a fusion protein, said fusion protein comprising an apoplast or plastid signal peptide, a SUMO protein or a C-terminal domain of a SUMO protein, and a zymogen of said protease.

The invention also provides a, preferably isolated, vector or nucleotide sequence comprising a coding sequence encoding a fusion protein, said fusion protein comprising an apoplast or plastid signal peptide, a SUMO protein or derivative of a SUMO protein, and a zymogen of said protease.

The present invention also provides a transgenic plant or plant cells for expressing a protease or a fusion protein comprising said protease, said plant comprising a nucleotide sequence containing a promoter active in vegetative tissue and, downstream of said promoter and operably linked thereto, a coding sequence encoding a fusion protein comprising a plastid or apoplast signal peptide, a SUMO protein or a derivative thereof, and a zymogen of said protease.

The present invention also provides a use of a SUMO protein or a derivative of a SUMO protein for expressing a protease or a zymogen of a protease in a plant or in plant cells.

The inventors have surprisingly found that proteases that are potentially toxic to plant tissue can be efficiently expressed in plants when targeted to the apoplast or to plastids as a fusion with a SUMO protein (or a derivative thereof). Since the SUMO protein (or a derivative thereof) can be cleaved off from the fusion protein when the zymogen of the protease is converted to the protease, no additional working step for removing the SUMO protein from the protease of interest is necessary, whereby the process of the invention is advantageous and convenient for the downstream isolation and purification of the protease of interest.

Transgenic corns seeds used in the prior art for protease production in plants are a problematic production system, since it is almost impossible to avoid contamination of non-GM corn seeds with traces of transgenic corn seeds in industrial scale agriculture. Further, the expression yield obtained for trypsin in corn seeds is not satisfactory, resulting in high costs of trypsin produced by this method. The inventors of the present invention have surprisingly found that it is possible to obtain a high expression yield of proteases in plant leaves although protease inhibitors (such as trypsin inhibitor) reducing a proteolytic degradation of plant host tissue expressing the protease were not expected to a significant extent in leaf tissue. Contrary to what could be expected from Woodard et al. (2003, Biotechnol. Appl. Biochem., 38 123-130), vegetative (or green) tissue such as leaf material has been found to be an ideal tissue for expressing proteases in plants in a high yield.

The process of the invention is not limited to maize but can be performed in plants such as Nicotiana species that are not part of the human food chain. Further, it is not necessary to harvest seeds or transgenic plants for isolating the protease. Instead, the invention can be performed by harvesting plants having expressed protease before seeds have reached a viable growth state. Thereby, contamination of non-GM corn seeds by transgenic seeds can be effectively avoided. In one embodiment, the invention allows it to avoid the use of transgenic plants by using transient expression of the protease of interest. In contrast, transient expression is difficult to practice if a protease is expressed in corn seeds as described by Woodard et al.

The process of the invention can be performed by transient expression or using transgenic plants. Transgenic plants for the invention contain in a nuclear genome the heterologous nucleotide sequence of the invention. In a transient expression system, said nucleotide sequence is provided to a plant, whereby production of said protease may be triggered by the provision of said nucleotide sequence to a plant. The nucleotide sequence is typically not incorporated into the genome of the plant host when the process of the invention is performed by transient expression. In any event, in a transient expression process, plants or plant cells having incorporated the nucleotide sequence into their genome are not selected, e.g. by using an antibiotic resistance marker, from plants or cells not having the nucleotide sequence incorporated into the genome. As a consequence, plants or plant cells having incorporated the nucleotide sequence of the invention into their genome are not produced to a significant extent, whereby transmission of the nucleotide sequence of the invention to progeny plants and seeds is very unlikely. If the process of the invention is carried out by transient expression, the nucleotide sequence of the invention may be provided to a plant having reached a desired growth state.

Said nucleotide sequence of the invention is heterologous, since it does not naturally occur in genome of the plant, or cells thereof, used in the process of the invention.

The nucleotide sequence of the invention comprises the coding sequence of the invention. The coding sequence encodes the fusion protein of the invention. In addition to said coding sequence, the nucleotide sequence of the invention may further have genetic elements for expressing said coding sequence into said fusion protein. Examples of such genetic elements are a promoter active in said plant and a transcription termination region. Said promoter is operably linked to said coding sequence such that expression of the coding sequence is under the control of the promoter. The promoter may be a constitutive promoter active in said plant such as the CaMV 35S promoter. Alternatively, said promoter may be an inducible promoter so that production of said protease can be induced at will, such as at a desired growth state of the plant. In a further alternative, said nucleotide sequence may be incorporated into a chromosome of said plant such that expression of said coding sequence is possible under the transcriptional control of a native host promoter as described in WO 02/46440.

In one embodiment, said nucleotide sequence is or encodes a replicon comprising said coding sequence encoding said fusion protein. Herein, a replicon is a nucleic acid capable of replicating independently from the plant nuclear replication machinery. For this purpose, said replicon has an origin of replication that can be recognized by a nucleic acid polymerase that is present in or that is provided to cells of said plant. The replicon may be a DNA replicon or an RNA replicon. In one embodiment, the replicon is an RNA replicon. In another embodiment, said replicon is a viral replicon. “Viral” means that the replicon contains, e.g. for replicating the viral replicon, one or more sequence portions of a length of at least 5, preferably at least 10 more preferably of at least 20 contiguous nucleotides, or one or more genetic element derived from a virus. Examples of such genetic elements are an origin of replication recognised by the viral nucleic acid polymerase. The viral replicon may, but does not have to, encode the nucleic acid polymerase of the virus. “Derived” means that the sequence portion or genetic element is taken from a virus or is a DNA copy of a sequence portion taken from an RNA virus. The viral replicon may be a DNA viral replicon or an RNA viral replicon. In an advantageous case, the replicon is an RNA viral replicon. Viral RNA replicons generally use viral polymerases for replicating the replicon; polymerases native to the plant host generally cannot replicate viral RNA replicons. RNA viral replicons further use an origin of replication that is not recognized by native plant polymerases. The viral polymerase may be encoded on the RNA replicon or may be provided in trans from a separate vector or from a transgene encoding such viral polymerase whereby the transgene is incorporated into a nuclear or organellar genome of the plant.

Said nucleotide sequence may be or may encode an RNA replicon such as a viral RNA replicon. Said viral RNA replicon may use the replication and expression machinery of a natural plant RNA virus. Suitable RNA viruses from which RNA replicons of the invention may be built on are, for example, positive-sense single-stranded plant RNA viruses. Examples of such plant RNA viruses are tobamoviruses such tobacco mosaic virus, crucifer-infecting tobamovirus, or turnip vein clearing virus. These viruses and their use for expressing a protein of interest in plants are known (see below). Thus, said RNA replicon may encode an RNA-dependent RNA polymerase (“replicase”) capable of replicating said RNA replicon. Further, the RNA replicon will contain an origin of replication that is recognized by the replicase.

There are various ways how a plant or plant cells can be provided with the replicon of the invention. In one embodiment, the nucleotide sequence of the invention is said replicon, and the plant or plant cells are infected directly with the replicon.

In another embodiment, the plant or plant cells are provided with a nucleotide sequence encoding said replicon. If said replicon is an RNA replicon, the nucleotide sequence may be a DNA nucleotide sequence encoding said RNA replicon. The DNA nucleotide sequence may have a promoter for producing said RNA replicon by transcription of a portion of said nucleotide sequence encoding said replicon, e.g. by a native RNA polymerase of the plant or plant cell.

A DNA nucleotide sequence encoding an RNA replicon may be incorporated into the nuclear genome of a plant or plant cells, whereby a transgenic plant or plant cells, respectively, are produced. The promoter present in the DNA nucleotide sequence may be an inducible promoter. In this way, formation of the replicon from which said fusion protein is expressed can be triggered at will by inducing the inducible promoter. Suitable inducible promoters are known in the art. Said inducible promoter may be part of an alcohol inducible system as described in example 4. Measures to suppress the consequences of any leaky expression by an inducible promoter system may be taken, e.g. as described in WO 2007/137788 that is incorporated herein in its entirety. This embodiment may be used together with transgenic plants containing said nucleotide sequence integrated into a nuclear or organellar chromosome.

In a transient expression method of the invention, a plant may be provided with a replicon. Similarly as described above, said replicon may be a DNA or an RNA replicon such as a viral DNA replicon or a viral RNA replicon. Cells of said plant may be provided with said replicons directly, such as by infecting said plant with said replicon or by particle bombardment using particles coated with said replicon. Alternatively, a plant may be provided with a replicon indirectly, such as by Agrobacterium-mediated transfection using Agrobacteria containing Ti plasmids containing T-DNA comprising a nucleotide sequence encoding said replicon (“agroinfection”). After having entered cells of said plant, the DNA or RNA replicon can be activated from said T-DNA by transcription by a DNA-dependent polymerase such as a DNA-dependent polymerase that is native to said plant. For this purpose, the T-DNA typically contains a promoter upstream of the nucleic acid encoding the replicon. After formation of the replicon in cells of said plant, the replicon replicates and expresses said coding sequence for producing the fusion protein of the invention. Agroinfection may be performed using highly diluted suspensions of Agrobacterium as described in WO 2006/3018, notably page 12 bottom to page 13, middle and page 39.

In an advantageous transient expression method, the replicon is an RNA replicon and said plant is provided with said RNA replicon by transforming said plant or said plant cells with a DNA vector as a DNA nucleotide sequence encoding said RNA replicon. Alternatively, said plant or said plant cells are provided with two or more DNA vectors encoding together said RNA replicon, whereby a DNA nucleotide sequence encoding an RNA replicon may be generated inside cells of said plant e.g. by site-specific DNA recombination as described in WO 02/88369. After recombination, the RNA replicon may be formed by transcription involving a native plant host RNA polmyerase and a promoter present on the DNA nucleotide sequence.

The coding sequence of the invention encodes the fusion protein of the invention, said fusion protein comprising at least the following three fusion protein segments: (i) an apoplast or plastid signal peptide, (ii) a SUMO protein or a derivative of a SUMO protein, and (iii) a zymogen of said protease. These elements of said fusion protein may be arranged in different orders. However, the signal peptide is preferably placed at the N-terminal end of the fusion protein in order to be functional for targeting the fusion protein to the apoplast or into the plastids. The zymogen of said protease may be placed at the C-terminus of the fusion protein, which allows its easy separation from the remainder of the fusion protein by a proteolytic cleavage at a single peptide bond. Thus, in one embodiment, the order of the essential fusion protein segments (i) to (iii) may be, in N-terminal to C-terminal direction, as listed in claim 1.

The fusion protein of the invention may further contain a polypeptide segment usable as a purification tag for facilitating the purification of the fusion protein or the protease of interest. The purification tag may be located at an internal position of said fusion protein such as on the C-terminal side of the signal peptide and on the N-terminal side of the zymogen of the protease. However, other orders are also possible e.g. placing the purification tag at the C-terminus of the SUMO protein. Purification tags that can be used for practicing this invention include, but are not limited to: FLAG tag, polyhistidine tags, polyarginine tags, influenza virus HA tag, GST-tag, protein A tag, maltose binding protein (MBP), S-tag, the AviD tag, etc.

The protease to be produced according to the invention may be any protease that is naturally expressed as a zymogen that is activated by proteolytic cleavage to produce the active protease. Such proteases are known in the art. Chymotrypsin and trypsin are examples of proteins to be produced by the invention. The zymogens of chymotrypsin and trypsin are chymotrypsinogen and trypsinogen, respectively. Other examples may include but are not limited to precursors (zymogens) of barley protease EPB2 indicated for celiac sprue treatment (Mikkonen et al., 1996, Plant Mol Biol., 31, 239-254; Vora et al., 2007, Biotechnol Bioeng., 98, 177-185.), elastase, caspases, carboxypeptidase A, thrombin and other proteases.

Plastid signal peptides (also referred to as “plastid transit peptides” in the art) for targeting proteins into plastids are known in the art. Examples of plastig signal peptides are found in WO 2004/101797. Signal peptides for targeting proteins into the secretory pathway and into the apoplast are also known from general knowledge. The signal peptides described in WO 02/101006 may be used for targeting the fusion protein to the apoplast.

Said fusion protein further comprises a SUMO protein or a derivative of a SUMO protein. SUMO proteins usable in the present invention are SUMO proteins that were used in the prior art in bacterial protein expression systems, cf. Butt et al., 2005, Protein Expr Purif., 43, 1-9; Marblestone et al., 2006, Protein Sci., 15, 182-189; Su et al., 2006, Protein Pept. Lett., 13, 785-792; Weeks et al., 2007, Protein Expr Purif., 53, 40-50; US2004018591; EP1654379).

SUMO (small ubiquitin-like modifier) is a member of the superfamily of ubiquitin-like polypeptides (Melchior, F., 2000, Annu. Rev. Cell. Dev. Biol., 16, 591-696; Schwartz & Hochstrasser, 2003, Trends Biochem. Sci., 28, 321-328; Dohmen, R J., 2004, Biochim. Biophys. Acta, 1695, 113-131; Hay, R T., 2007, Trends Cell Biol., 17, 370-376). All SUMO proteins contain a ubiquitin domain (outlined in FIG. 10A) and are about 100 amino acid residues in length (usually within the range of 90 and 115). Alignments of different SUMO proteins from different organisms including plant SUMO proteins are shown in FIG. 10.

In higher plants several genes were identified that encode different SUMO proteins. The genes are designated SUMO1, SUMO2, SUMO3, SUMO4, SUMO5, SUMO6, SUMO7, SUMO8 and SUMO9 in Arabidopsis, but only four of them (SUMO1, SUMO2, SUMO3 and SUMO5) were found to be transcriptionally active (Kurepa et al., 2003, J. Biol. Chem., 278, 6862-6872). Among these SUMO proteins, the fusion with SUMO1 generally gives the highest expression levels for protein of interest in the present invention. In the present invention, a SUMO protein known from an organism such as a plant, an animal or yeast may be used, whereby plant SUMO proteins are generally preferred.

Alternatively, derivatives of a natural SUMO protein may be used. A derivative of a SUMO protein herein comprises, in one embodiment, at least 50 contiguous amino acid residues, in another embodiment at least 60 contiguous amino acid residues, in a further embodiment at least 70 contiguous amino acid residues, and in a still further embodiment at least 80 contiguous amino acid residues.

A SUMO protein-derivative according to the invention is characterized by comprising the typical consensus sequence of a SUMO protein. Such consensus sequence can be determined by making sequence alignments of known SUMO proteins. The consensus sequence defines the a specific amino acid residue or a selection of specific amino acid residues for certain amino acid residue positions, whereas any desired amino acid residue can be chosen at other positions with little influence on the expression properties of the protease to be expressed according to the invention. Suitable consensus sequences can be defined at varying degrees of specificity. In the broadest sense, the derivative of a SUMO protein has the consensus sequence: -L/F/M-X₁₉-F/I-X₃-G/D-X₇-T/S-P/A-.

In other embodiments of the invention, a derivative of a SUMO protein has any one of the following amino acid sequences:

-L/F/M-X₁₉-F/I-X₃-G/D-X₇-T-P-;
-L-(X)₁₉-F-X₃-G-X₇-T-P-;
-L-X₁₉-F-X₃-G-X₇-T-P-X₁₈-G-G-;
-L/I-X-V/L-Xₐ-L-X₁₉-F-X₃-G-X₇-T-P-;
-L-X-K/R-X₁₇-F-X₃-G-X₇-T-P-;
-L-X-K/R-L/M-X₁₆-F-X₃-G-X₇-T-P-;
-L-X-K/R-L/M-M-X₁₅-F-X₃-G-X₇-T-P-;
-L-X-K/R-L/M-M-X₁₅-F-X₃-G-X₇-T-P-X₁₈-G-G-;
-L-X-K/R-L/M-M-X₁₅-F-X₃-G-X₇-T-P-X₂-L-;
-L-X-K/R-L/M-M-X₁₅-F-X₃-G-X₇-T-P-X₂-L-D/E-X₂-D/E-;
-L-X-K/R-L/M-M-X₁₅-F-X₃-G-X₇-T-P-X₂-L-D/E-X₂-D/E-
X₃-I-D/E-;
-L-X-K/R-L/M-M-X₁₅-F-X₃-G-X₇-T-P-X₂-L-D/E-X₂-D/E-
X₃-I-D/E-X₆-G-G-;
-L/I-X-V/L-Xₐ-L-X-K/R-L/M-M-X₁₅-F-X₃-G-X₇-T-P-X₂-
L-D/E-X₂-D/E-X₃-I-D/E-X₆-G-G-;
-L-K-V-K-X_{b}-L-X₁₉-F-X₃-G-X₇-T-P-;
-L-K-V-K-X_{b}-L-X-K-X-M-X₁₅-F-X₃-G-X₇-T-P-;
-L-K-V-K-X_{b}-L-X-K-X-M-X₂-Y-X₁₂-F-X₃-G-X₇-T-P-;
-L-K-V-K-X_{b}-L-X-K-X-M-X₂-Y-X₁₂-F-X₃-G-X₇-T-P-X₂-
L-;
-L-K-V-K-X_{b}-L-X-K-X-M-X₂-Y-X₁₂-F-X₃-G-X₇-T-P-X₂-L-
X₁₅-G-G-;
-L-K-V-K-X_{b}-L-X-K-X-M-X₂-Y-X₁₂-F-X₃-G-X₇-T-P-X₂-L-
X₇-I-;
-L-K-V-K-X_{b}-L-X-K-X-M-X₂-Y-X₁₂-F-X₃-G-X₇-T-P-X₂-L-
X₅-X-I-;
-L-K-V-K-X_{b}-L-X-K-X-M-X₂-Y-X₁₂-F-X₃-G-X₇-T-P-X₂-L-
X₇-I-X₇-G-G-;
-L-K-V-K-X-Q-X_{c}-L-X-K-X-M-X₂-Y-X₁₂-F-X₃-G-X₇-T-P-
X₂-L-X₇-I-X₇-G-G-;
-L-K-V-K-X_{b}-L-L/K-K-L/M-M-X₂-Y-X₁₂-F-X₃-G-X₇-T-P-
X₂-L-X₇-I-X₇-G-G-;
-L-K-V-K-X-Q-X_{c}-L-L/K-K-L/M-M-X₂-Y-X₁₂-F-X₃-G-X₇-
T-P-X₂-L-X₇-I-X₇-G-G-;
-L-X-K/R-L-X₁₆-F-X₃-G-X₇-T-P-;
-L-X-K/R-L-M-X₁₅-F-X₃-G-X₇-T-P-;
-L-X-K/R-L-M-X₅-R/K-Q/R-X₈-F-X₃-G-X₇-T-P-;
-L-X-K/R-L-M-X₅-R-Q-X₈-F-X₃-G-X₇-T-P-;
-L-X-K/R-L-M-X₅-R-Q-X₈-F-X₃-G-X₇-T-P-X₂-L-X-M-;
-L-X-K/R-L-M-X₅-R-Q-X₃-M-X₄-F-L-X₂-G-X₇-T-P-X₂-L-
X-M-;
-L-X-K/R-L-M-X₅-R-Q-X₈-F-L-X₂-G-X₇-T-P-X₂-L-X-M-E-
X₄-I-X₇-G-G-:
-L-X-K/R-L-M-X₅-R-Q-X₈-F-L-X₂-G-X-R-X₅-T-P-X₂-L-X-
M-E-X₄-I-X₇-G-G-:

In the above sequences of a SUMO protein derivative of the invention, the amino acid consensus sequences are given in N-terminal to C-terminal direction;

a is an integer of 17 or 18;

b is an integer of 16 or 17;

c is an integer of 14 or 15;

each letter stands for an amino acid residue;

X stands for any amino acid residue;

letters other than X stand for amino acid residues in the standard one-letter code;

a numerical subscript to a letter indicates that the amino acid residue defined by said letter is present contiguously and connected by peptide bonds as many times as indicated by the numerical value of the subscript;

“-” stands for a peptide bond connecting adjacent amino acid residues; and

“/” indicates that the amino acid position defined by two consecutive “-” can be occupied by any of the amino acid residues defined by letters separated by “/”.

After having expressed the fusion protein of the invention from said coding sequence, said protease or said zymogen or a fusion protein comprising said zymogen can be isolated from vegetative tissue such as from leaf tissue of said plant and purified according to standard methods of protein purification. If the process of the invention is performed in transgenic plants, the protease or the zymogen or a fusion protein comprising the zymogen are preferably isolated before viable seeds have developed from said plant in order to prevent contamination of non-transgenic seeds with transgenic seeds and in order to avoid spread of transgenic seeds in the environment.

Isolation typically includes the following steps: homogenising the tissue containing expressed protease or fusion protein comprising the protease, extracting the protease or fusion protein comprising the protease into a solvent (usually an aqueous, buffered solvent), and separating cell debris and other material insoluble in the solvent e.g. by centrifugation or filtration. The protease or fusion protein comprising the protease may then be purified from other components derived from the tissue present in the solvent. Purification methods established for the protease or fusion protein to be purified may be used. If the fusion protein contains an affinity tag, purification may include affinity chromatography.

If the isolated protein is not the activated protease but the zymogen of the protease or a fusion protein comprising said zymogen, the active protease may be generated from said zymogen or from said fusion protein by proteolytic cleavage. Said proteolytic cleavage may be achieved by a protease recognizing the cleavage site of said zymogen. If the isolated protein is or comprises trypsinogen, the protease enterokinase may be used for activating trypsin from trypsinogen. Chymotrypsin may be activated from chymotrypsinogen by trypsin.

The present invention can be performed with any plant or cells thereof. It is preferred that the invention is performed with plants. Among plants, higher plants are preferred. Among higher plants, the invention may be performed with monocot or with dicot plants. Plants that are not part of the human food chain are preferred. Examples of plants that may be used in the invention are Nicotiana species such as Nicotiana benthamiana and Nicotiana tabacum.

### Advantageous Embodiments

A process of producing a protease in a plant or in plant cells, comprising
(a) providing a plant comprising a heterologous nucleotide sequence comprising a coding sequence encoding a fusion protein, said fusion protein comprising preferably in the following order (i) to (iii) in N-terminal to C-terminal direction:
   (i) an apoplast or plastid signal peptide;
   (ii) a SUMO protein or a derivative of a SUMO protein; and
   (iii) a zymogen of said protease, and
(b) expressing said fusion protein,
   said derivative of a SUMO protein comprising the consensus sequence -L/F/M-X₁₉-F/I-X₃-G/D-X₇-T/S-P/A-.

A process of producing a protease in a plant or in plant cells, comprising
(a) providing a plant comprising, on a nuclear chromosome, a heterologous nucleotide sequence comprising a coding sequence encoding a fusion protein, said fusion protein comprising preferably in the following order (i) to (iii) in N-terminal to C-terminal direction:
   (i) an apoplast or plastid signal peptide;
   (ii) a SUMO protein or a derivative of a SUMO protein; and
   (iii) a zymogen of said protease, and
(b) expressing said fusion protein,
   wherein
   said nucleotide sequence comprising an inducible promoter upstream of said coding sequence and operably linked to said coding sequence, and said derivative of a SUMO protein comprising the consensus sequence -L/F/M-X₁₉-F/I-X₃-G/D-X₇-T/S-P/A-.

A process of producing a protease in a plant or in plant cells, comprising providing a plant with an RNA replicon comprising a coding sequence encoding a fusion protein, said fusion protein comprising preferably in the following order (i) to (iii) in N-terminal to C-terminal direction:
(i) an apoplast or plastid signal peptide;
(ii) a SUMO protein or a derivative of a SUMO protein; and
(iii) a zymogen of said protease;
   wherein said plant is provided with said RNA replicon by transforming said plant with a DNA vector encoding said RNA vector; and said derivative of a SUMO protein comprises the consensus sequence -L/F/M-X₁₉-F/I-X₃-G/D-X₇-T/S-P/A-. Said DNA vector may contain Agrobacterial T-DNA encoding said RNA replicon and having a promoter for generating said RNA replicon in cells of said plant by transcription.

A process of producing a protease in a plant or in plant cells, comprising providing, by Agrobacterium-mediated transformation, a plant with an RNA replicon comprising a coding sequence encoding a fusion protein, said fusion protein comprising in the following order (i) to (iii) in N-terminal to C-terminal direction:
(i) an apoplast or plastid signal peptide;
(ii) a SUMO protein or a derivative of a SUMO protein; and
(iii) a zymogen of said protease.

A process of producing a protease of interest in a plant, comprising:
(A) providing a plant comprising:
   a heterologous nucleotide sequence encoding an RNA replicon and comprising an inducible promoter operably linked to a sequence encoding said RNA replicon;
   said RNA replicon not encoding a protein providing for cell-to-cell movement of said RNA replicon in said plant;
   said RNA replicon encoding a polymerase being adapted for replicating said RNA replicon; said RNA replicon comprising a coding sequence encoding a fusion protein, said fusion protein comprising, preferably in the following order (i) to (iii) in N-terminal to C-terminal direction, an apoplast or plastid signal peptide, a SUMO protein or a derivative of a SUMO protein, and a zymogen of said protease; and
(B) inducing, in said plant or plant cells of step (A), said inducible promoter, thereby producing said protease or a fusion protein comprising said protease of interest in said plant or in cells of said plant.

Optionally, said plant may comprise a second heterologous nucleotide sequence comprising a nucleotide sequence encoding a protein enabling cell-to-cell movement of said RNA replicon, wherein said second heterologous nucleotide sequence comprises a second inducible promoter operably linked to said nucleotide sequence encoding said protein enabling cell-to-cell movement of said RNA replicon; said inducible promoter and said second indubible promoter may be the same types of inducible promoters such as promoters of an alcohol inducible system.

Other embodiments described herein may be combined with the above advantageous embodiments. One such embodiment is the combination of trypsin as the protease to be produced and a Nicotiana plant such as a tobacco plant.

### BRIEF DESCRIPTION OF THE FIGURES

FIG. 1 shows cDNA and protein sequences for (A) bovine pancreas cationic pretrypsinogen and (B) codon-optimised cDNA (synthesised by GENEART AG, Regensburg, Germany) and protein sequences of barley EPB-2 protease precursor. Coding sequences for EPB2 protease precursor are shown in bold. Sequences recognized by site-specific restriction enzyme BsaI are shown in italic and underlined. The protein sequence in FIG. 1(A) is SEQ ID NO: 4. The nucleic acid in FIG. 1(A) is SEQ ID NO: 5. The nucleic acid in FIG. 1(B) is SEQ ID NO: 6. The protein sequence in FIG. 1(B) is SEQ ID NO: 7.

FIG. 2 depicts the cloning strategy for trypsinogen and trypsin genes. NTR—viral 3′ non-translated region; 3′ NOS—transcription termination region of nopaline synthase gene; pNOS—promoter of nopaline synthase; NPTII—neomycin phosphotransferase II gene, AttB—recombination site recognised by site-specific integrase phC31. The sequences of primers bovp1 to bovp9 are shown. Primers bovp1 to bovp9 are SEQ ID NOs: 8 to 16, respectively.

FIG. 3 depicts T-DNA regions of the binary vectors pICH29090, pICH29373 (pICH29377—the same as pICH29373, but different clone), pICH21825, pICH21811, pICH18812 and 14011. pAct2—transcription promoter of Arabidopsis ACTIN2 gene; TVCV polymerase—RNA-dependent RNA polymerase of Turnip Vein Clearing Virus with introns indicated by dotted portions; MP—viral movement protein with introns indicated by dotted portions; NTR —viral 3′ non-translated region; 3′ NOS—transcription termination region of nopaline synthase gene; SUMO1—coding sequence with introns (indicated by dotted portions) of Arabidopsis SUMO1 gene; pNOS—promoter of nopaline synthase gene; pHSP81.1—promoter of the gene for Arabidopsis heat-shock protein HSP81.1; phC31—site-specific integrase of phage C31; NLS—nuclear localization signal; AttP and AttB—recombination sites recognised by site-specific integrase phC31; SP—apoplast targeting signal peptide (rice amylose); dotted segments stand for introns.

FIG. 4 shows results of expression of apoplast-targeted recombinant SUMO-trypsinogen fusion in N. benthamiana leaves using plant viral vectors.
A—Coomassie-stained polyacryamide gels; B—Western blot with anti-trypsinogen antibodies (1:3000 dilution; 5 min exposure); C—testing for Trypsin enzymatic activity by using milk assay.
dpi—days post-innoculation; M—molecular weight markers; U—uninfected tissue (control).

FIG. 5 shows expression of chloroplast-targeted recombinant SUMO-trypsinogen fusion in N. benthamiana leaves using plant viral vectors. Overnight cultures of agrobacteria for infiltration were 1:10 diluted. Tissue was harvested at between 6 and 13 dpi (days post infection), extracted with 6 volumes 1× Laemmli buffer and boiled before centrifugation. 0.05 ml/slot of supernatant was loaded onto the polyacrylamide gel.

FIGS. 6A and B show the kinetics of BAPNA cleavage with trypsin formed after trypsinogen processing with enterokinase. TK—trypsinogen control (graphic 1), EK—enterokinase control (graphic 2); Numbers 5; 2; 1; 0,5; 0,2 and 0,1 (graphics 3 to 8, respectively) are the respective concentrations (in μg/ml) of enterokinase cleaved trypsinogen standards; FA+EK—folding reaction with enterokinase (graphics 8 and 10); FA ohne EK—folding reaction without enterokinase (graphics 9 and 11.

FIG. 7 shows expression of apoplast-targeted recombinant SUMO-trypsinogen fusion in stably transformed N. benthamiana plants carrying plant viral vector under control of inducible promoter.
A—T-DNA regions of the binary vectors pICH26505, pICH18693 and pICH28287
TVCV polymerase—RNA-dependent RNA polymerase of Turnip Vein Clearing Virus with introns indicated by dotted portions; MP—viral movement protein with introns indicated by dotted portions; NTR—tobamoviral 3′ non-translated region; 3′ NOS—transcription termination region of nopaline synthase gene; SUMO1—coding sequence with introns (indicated by dotted portions) of Arabidopsis SUMO1 gene; pNOS—promoter of nopaline synthase gene; pAlcA—inducible promoter of inducible A. nidulans alcA gene encoding alcohol dehydrogenase; alcR—transcriptional activator of the alc regulon of Aspergillus nidulans; p35S—35S promoter of CaMV; NPTII—neomycin phosphotransferase II gene.
B—coomassie-stained polyacrylamide gel (left) and Western blot analysis (right) of total soluble protein extracted from transgenic plants transformed with pICH28287.
N2, N3 and N4—different transgenic N. benthamiana plants; ni—total soluble protein extracted from not induced (infiltrated) plant material; inf—total soluble protein extracted 7 days after infiltration of plants with pICH26505, pICH18693 and 2% ethanol; s, s1 and s2—commercially available trypsin loaded at the concentrations 1.3 microgram, 50 nanogram and 100 nanogram, respectively.
C—test for trypsin activity using digestion of milk proteins. nc—negative control (no trypsin was added); pc—positive control (1 μl of commercially available bovine trypsin from ICN Biomedicals, CA, USA, 1 mg/ml in 1 mM HCl).

FIG. 8 depicts T-DNA regions of the binary vectors pICH28575, pICH29392, pICH24200, pICH28512 and pICH28644. Cloning strategy for codon-optimized EPB2 zymogen resulting in pICH29392 3′ provector is shown in upper part of the figure. pAct2—transcription promoter of Arabidopsis ACTIN2 gene; TVCV polymerase—RNA-dependent RNA polymerase of Turnip Vein Clearing Virus with introns indicated by dotted portions; MP—viral movement protein with introns indicated by dotted portions; NTR—viral 3′ non-translated region; 3′ NOS—transcription termination region of nopaline synthase gene; SUMO1—coding sequence with introns of Arabidopsis SUMO1 gene; pNOS—promoter of nopaline synthase gene; AttP and AttB—recombination sites recognised by site-specific integrase phC31.

FIG. 9 shows the expression of apoplast- and chloroplast-targeted recombinant SUMO-EPB2 zymogen fusions in N. benthamiana leaves using plant viral vectors. Plant tissue was harvested 8 dpi (days post infection), extracted with 10 (w/v) volumes of 1× Laemmli buffer or 5 (w/v) of tris extraction buffer and boiled before centrifugation. The 0.05 ml/slot of supernatant was loaded onto the polyacrylamide gel. In case of tris extraction buffer supernatant before loading was diluted with equal volume of 2× Laemmli buffer. Expression of EPB2 (pICH29392) with: (1) barley apoplast targeting signal peptide (pICH24200); (2) its own (EPB2) apoplast targeting signal peptide (pICH28512); (3) EPB2 apoplast targeting signal peptide and prosequence (pICH28644); (4) chloroplast targeting transit peptide-SUMO fusion (pICH21811); (5) apoplast targeting signal peptide-SUMO fusion (pICH21825); F1—Yersinia pestis F1, expressed with barley apoplast targeting signal peptide; NC—negative control (protein extracted from not infiltrated plant leaf material). The positions of mature EPB2 protein and SUMO1-EPB2 zymogen fusions on Coomassie-stained gels are elipsed. Western blot analysis (lower panel) was performed with 1000× diluted anti-EPB2 antibodies.

FIG. 10 shows multiple alignments of sequences from different SUMO proteins. The program used: AlignX (Vector NTI Suite 7.1), based on the ClustalW algorithm.
(A)—alignment of ten different SUMO protein sequences of animal, yeast and plant origin.
Mus musculus: SMT3.2_MM—SUMO2 (Acc. No. NP_{—}579932): SEQ ID NO: 17;
Mus musculus SMT3.3_MM—SUMO3 (Acc. No. EDL31801): SEQ ID NO: 18;
Mus musculus SMT3_MM—SUMO1 (Acc. No. NP_{—}033486): SEQ ID NO: 19;
Oryza sativa: SMT3_OS—SUMO1 (Acc. No. P55857)): SEQ ID NO: 20;
Saccharomyces cerevisiae: SMT3_SC—SUMO1 (Acc. No. NP_{—}010798): SEQ ID NO: 21;
Arabidopsis thaliana: SUMO1_AT (Acc. No. P55852)): SEQ ID NO: 22;
SUMO2_AT (Acc. No. NP_{—}200327): SEQ ID NO: 23;
SUMO3_AT (Acc. No. NP_{—}200328): SEQ ID NO: 24;
SUMO4_AT (Acc. No. NP_{—}199683): SEQ ID NO: 25;
SUMO5_AT (Acc. No. NP_{—}565752): SEQ ID NO: 26;
Identities—4.1%; positives—61.8%. The ubiquitin domain is outlined.
(B) Alignment of five different Arabidopsis thaliana SUMO protein sequences SEQ ID NOs: 22 to 26 from top to bottom: Identity—13.9%, positives—66.4%.

FIG. 11 Continuation of FIG. 10. Alignment of SUMO1 protein sequences derived from mouse (Mus musculus): SEQ ID NO: 19,
rice (Oryza sativa): SEQ ID NO: 20;
yeast (Saccharomyces cerevisiae): SEQ ID NO: 21; and
arabidopsis (Arabidopsis thaliana): SEQ ID NO: 22;
Identity—26.4%; positives—92.5%.

FIG. 12 Map of pICH29090: sequence encoding for apoplast targeting signal peptide is shown in bold. The sequence of pICH29090 is shown in SEQ ID NO: 1. The sequence encoding the apoplast targeting signal peptide is shown in SEQ ID NO: 2.

FIG. 13 Map of pICH29373: the sequence of pICH29373 is identical to the one of pICH29090, except that the sequence encoding for apoplast targeting signal peptide of pICH29090 is replaced by the sequence encoding for artificial transit peptide shown in SEQ ID NO: 3.

FIG. 14 depicts the T-DNA regions of the binary vectors pICH20655, pICH21091, pICH21100, pICH21111, pICH21122, pICH21131, pICH20111, pICH7410 and pICH14011. ubi—coding sequences for Arabidopsis ubiquitin gene, the full length ubiquitin (ubiquitin, 76 aa) and different N-terminally truncated versions of ubiquitin having 61 aa, 42 aa and 33 aa) are shown in brackets; pICH21091pAct2—transcription promoter of Arabidopsis ACTIN2 gene; TVCV polymerase—RNA-dependent RNA polymerase of Turnip Vein Clearing Virus with introns indicated by dotted portions; MP—viral movement protein with introns indicated by dotted portions; NTR—viral 3′ non-translated region; 3′ NOS—transcription termination region of nopaline synthase gene; SUMO1, SUMO2—coding sequences with introns of Arabidopsis SUMO1 and SUMO2 genes; pNOS—promoter of nopaline synthase gene; AttP and AttB—recombination sites recognised by site-specific integrase phC31; pHSP81.1—promoter of N. tabacum gene encoding for heat-shock protein HSP81.1; NLS—nuclear localization signal.

FIG. 15 shows the N. benthamiana leaves infiltrated with different fusions of GFP 8 days after infiltration. Upper panel—infiltrated leaves under day light conditions; lower panel—under UV light. Control 10⁻³—expression of GFP alone, agrobacterial overnight culture was diluted 1000 folds before infiltration. pICH18971—integrase phi31 is under control of 35S promoter.

FIG. 16 shows the expression of different ubiquitin- and SUMO-GFP fusions in N. benthamiana leaves using plant viral vectors. Plant tissue was harvested 8 dpi (days post infection), extracted with 10 (w/v) volumes of 1× Laemmli buffer or 5 (w/v) of tris extraction buffer and boiled before centrifugation. The 0.05 ml/slot of supernatant was loaded onto the polyacrylamide gel. The gel was stained with Coomassie—blue after elecytrophoretic separation. The positions of GFP and GFP fusions are circled.

### DETAILED DESCRIPTION OF THE INVENTION

Ubiquitin fusion were previously suggested for augmenting protein expression in transgenic plants (Hondred et al., Plant Physiology 119 (1999) 713-723). However, when the inventors of the present invention tried to use ubiquitin fusions intended for large scale applications, it was found that the expression yields were small, which explains why very sensitive immoblot analysis had to be made by Hondred et al. for detecting expressed protein. The inventors have further found (example 6) that ubiquitin has toxic effects on plants, cf. FIGS. 15 and 16 showing necrosis on leaves and a generally low protein content at least with fusion proteins comprising full-length and the 31aa-truncated ubiquitin derivative. It was therefore highly surprising that other members of the ubiquitin family of proteins turned out to be not only non-toxic in plants when used for expressing fusion proteins, but provided excellent expression levels even when used for expressing toxic proteins like proteases. The process of the invention allows to achieve higher expression levels than the method of Woodard et al. (2003, Biotechnol. Appl. Biochem., 38, 123-130), and does not rely on transgenic seeds that are prone to contaminate seeds from non-transgenic plants and favor distribution of transgenic material in the environment.

It was previously shown that the expression level of recombinant proteins of interest can be improved via fusion with other proteins including SUMO (Butt et al., 2005, Protein Expr Purif., 43, 1-9; Marblestone et al., 2006, Protein Sci., 15, 182-189; Su et al., 2006, Protein Pept. Lett., 13, 785-792). Weeks et al., 2007, Protein Expr Purif., 53, 40-50; US2004018591; EP1654379). The above publications mostly relate to expression in E. coli. To the best of our knowledge, SUMO has so far not been used for improving expression levels of proteins of interest in plant expression systems, notably for cytotoxic proteins such as proteases.

The process of producing a protease of interest according to the invention involves expression of said protease of interest as a fusion protein which is compartmentalised within plant or plant cell by means of signal peptide or transit peptide-mediated targeting of the fusion protein. In the apoplast, the fusion protein may be processed to active protease, while in the chloroplast the fusion protein may be accumulated as protein inclusion bodies.

In the first step of the process of the invention, a plant or plant cells are transformed or transfected with a nucleotide sequence having a coding sequence encoding said fusion protein having a signal or transit peptide. Transformation may produce stably transformed plants or plant cells, e.g. transgenic plants. Alternatively, said plant or plant cells may be transfected for transient expression of said fusion protein. Several transformation or transfection methods for plants or plant cells are known in the art and include Agrobacterium-mediated transformation, particle bombardment, PEG-mediated protoplast transformation, viral infection etc.

Said nucleotide sequence may be DNA or RNA depending on the transformation or transfection method. In most cases, it will be DNA. In an important embodiment, however, transformation or transfection is performed using RNA virus-based vectors, in which case said nucleotide sequence is RNA.

Said nucleotide sequence comprises a coding region encoding a fusion protein. Said fusion protein comprises the SUMO protein. Said fusion protein further comprises a precursor of the protease of interest or zymogen that upon processing yields active protease (referred to as “protease of interest” in the following). Said protease of interest may be any protease that can be produced and isolated according to the process of the invention. It may be produced in an unfolded, misfolded or in a natural, functional folding state. The latter possibility is preferred.

Said fusion protein further comprises a signal peptide functional for targeting said fusion protein to the apoplast or for targeting said fusion protein to plastid. The apoplast targeting may be achieved with a signal peptide that targets the fusion protein into the endoplasmatic reticulum (ER) and the secretory pathway. All signal peptides of proteins known to be secreted or targeted to the apoplast may be used for the purposes of the invention. Preferred examples are the signal peptides of tobacco calreticulin, barley or rice amylase. Signal peptides that target a protein to plastids are also referred to as “transit peptides”. Any transit peptides can be utilized for practicing said invention. Preferred examples are artificial transit peptides or transit peptides of small subunit rubisco from tobacco. Other signal peptides for targeting the fusion protein to the apoplast are given in EP 1 423 524. For functional targeting the transit peptide or the signal peptide is positioned at the N-terminus of the fusion protein.

After the fusion protein has been expressed by a plant, the protease of interest or a fusion protein comprising the protease of interest can be isolated from the plant or plant cell by standard protein purification methods. The isolation of a protease of interest or a fusion protein thereof can be facilitated by incorporating a purification tag into the protease or fusion protein. Such systems are commercially available e.g. from Amersham Pharmacia Biotech, Uppsala, Sweden. A specific example frequently used for removing a His-tag is the factor Xa system.

In another embodiment of said invention, an isolated fusion protein can be processed for releasing the protease of interest (e.g. trypsin) from said fusion by using enterokinase that specifically cleaves the zymogen (trypsinogen) at the N-terminus, whereby the active protease (e.g. trypsin) is released.

Construction of the nucleotide sequence of the invention may be done according to standard procedures of molecular biology. The nucleotide sequence may contain a plant-specific promoter operably linked to said coding sequence and a transcription terminator after said coding sequence. In the case of stable transgenic plants, inducible expression of the fusion protein may be achieved if desired by an appropriately selected inducible expression system. In a preferred embodiment, virus-based vectors under control of alcohol-inducible system are used for performing this invention. Construction of such viral vectors are described in the reference examples and in the numerous publications.

The nucleotide sequence comprising the coding sequence encoding the fusion protein of the compartmentalized with the help of signal or transit peptide may be delivered into the plant cell preferably using a DNA or an RNA vector. The recombinant protein fusion is expressed and then targeted to the intercellular space (apoplast) in case of fusion with signal peptide or to plastid. The plants with said fusion protein may then be subjected to processing. Dependent from the form in which the protease is accumulated in the plant, the downstream processing might incorporate protein fusion refolding and cleavage in order to produce active protease (in case of plastids compartmentalization), or lead directly to the isolation of active protease (in case of apolplast targeting).

Various methods can be used to deliver the nucleotide sequence of the invention using a vector into the plant cell, including direct introduction of said vector into a plant cell by means of microprojectile bombardment, electroporation or PEG-mediated treatment of protoplasts (for review see: Gelvin, S. B., 1998, Curr. Opin. Biotechnol., 9 227-232; Hansen & Wright, 1999, Trends Plant Sci., 4, 226-231). Plant RNA and DNA viruses also present efficient delivery systems (Hayes et al., 1988, Nature, 334, 179-182; Palmer et al., 1999, Arch. Virol., 144 1345-1360; Lindbo et al., 2001, Curr. Opin. Plant. Biol., 4, 181-185). Vectors can deliver a transgene either for stable integration into the genome of the plant (direct or Agrobacterium-mediated DNA integration) or for transient expression of the transgene (“agroinfiltration”).

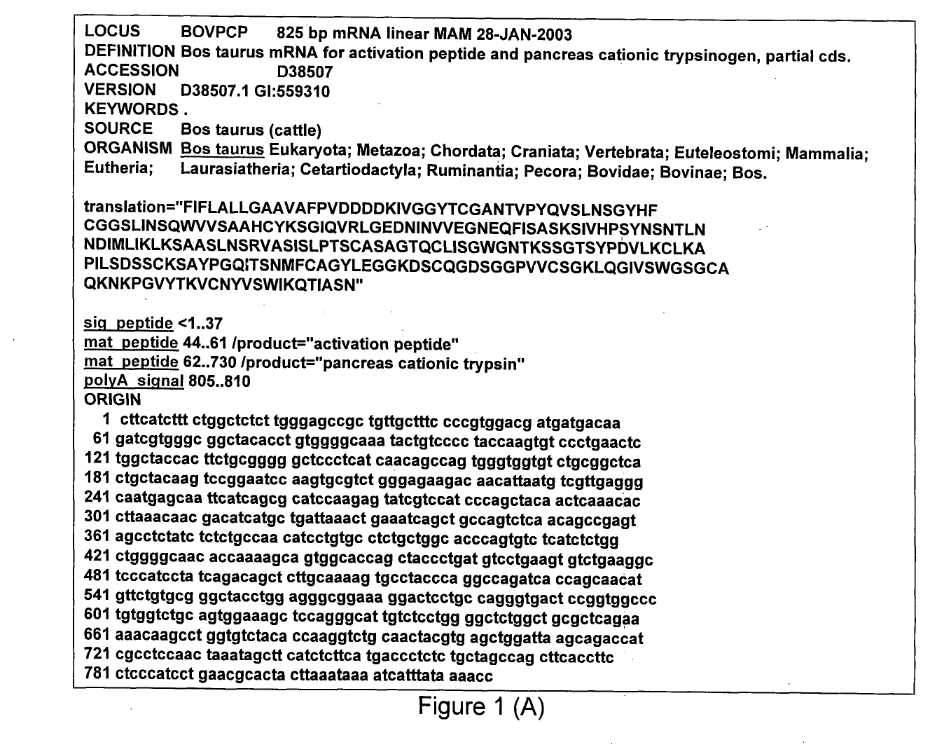

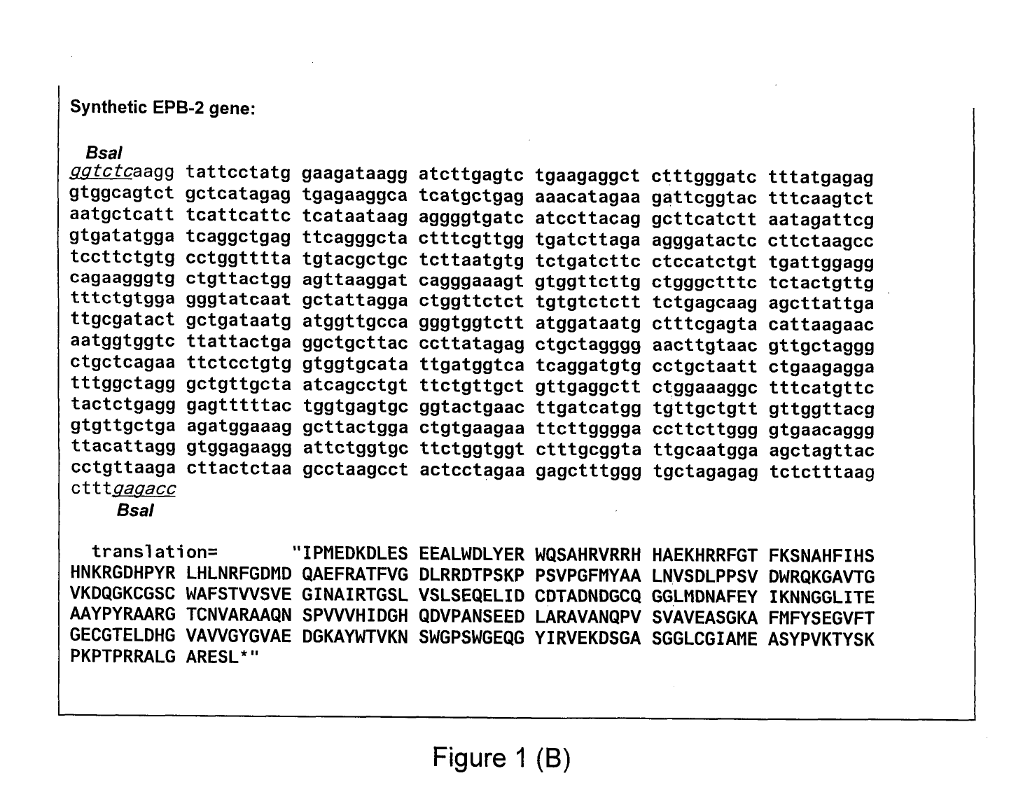

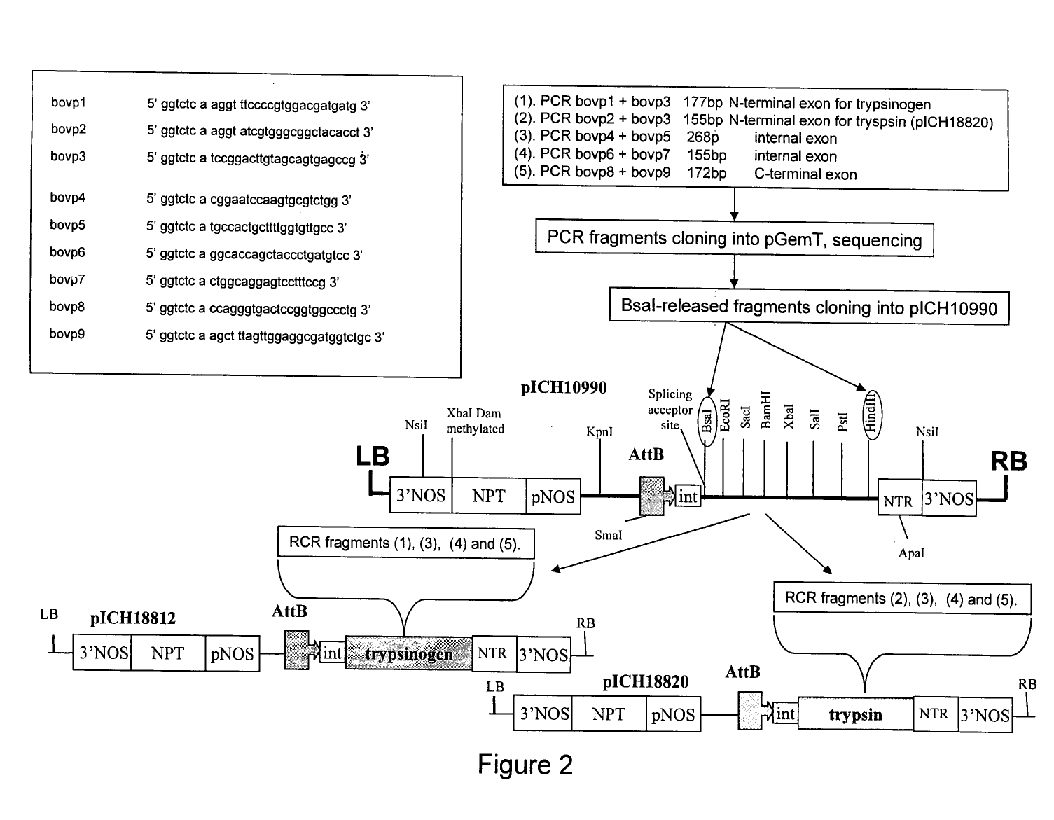

Different vectors may be used to express fusion protein in plant or plant cell. Suitable vectors for practicing said invention are the plant viral vectors. In one embodiment, RNA viral vectors are used. The use of such vectors for optimization of proteins expression and for large-scale production are described in detail in numerous publications (Marillonnet et al., 2004, Proc Natl Acad Sci USA, 101:6852-6857; Marillonnet et al., 2005, Nat Biotechnol., 23:718-723; Giritch et al.,2006, Proc Natl Acad Sci USA., 103, 14701-14706; Santi et al., 2006, Proc Natl Acad Sci USA., 103, 861-866). In one embodiment, cloning of a bovine trypsinogen gene (FIG. 1) into 3′ part of plant viral vector (3′ provector) is described (see example 1, FIG. 2). Such 3′ provector can be assembled into plant viral vector via site-specific recombination mediated by DNA recombinase (in said embodiment by phage C31 integrase). Using this approach, the 3′ provector carrying the coding sequence of the invention can be fused in frame to any other coding sequence of interest. The approach allows to optimize the expression level of recombinant protein of interest in the most convenient and speedy way.

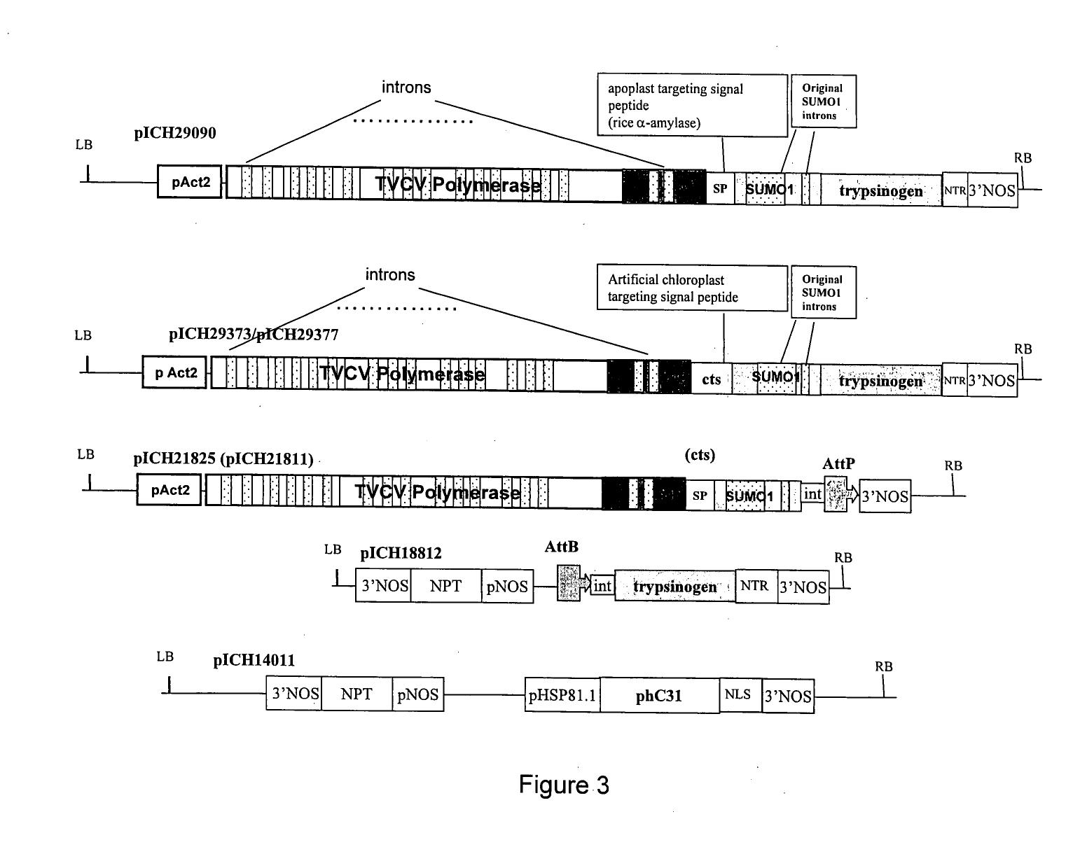

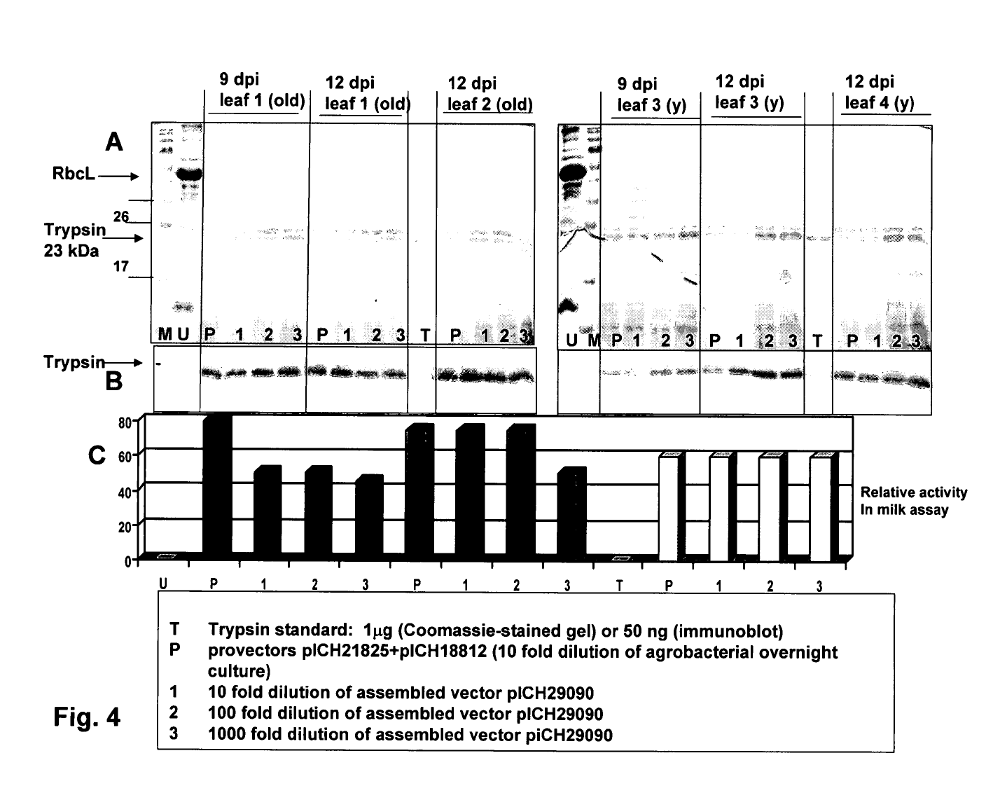

We have tested trypsinogen expression of many different fusions including translational fusions with five different A. thaliana SUMO proteins. The best results were obtained for SUMO1-trypsinogen fusion. Based on the results of studies with provectors, assembled plant viral vectors were designed for production of proteases in plant tissues. In FIG. 3 schematic representations of assembled viral vectors with trypsinogen targeted into apoplast (pICH29090) and chloroplast (pICH29373) are shown.

In yet another embodiment of this invention (example 2), the results of apoplast- and chloroplast-targeted trypsinogen expression using transient expression from provectors as well as from assembled viral vectors are described. It is evident from the results of protein (predominantly rubisco) degradation on coomassie-stained gel and protease activity measurement using milk assay (FIG. 4, A and C, respectively) that apoplast-targeting of trypsinogen leads predominantly to the formation of active trypsin. This finding is also confirmed by Western blot analysis with commercially available anti-trypsinogen antibodies (FIG. 4, B).

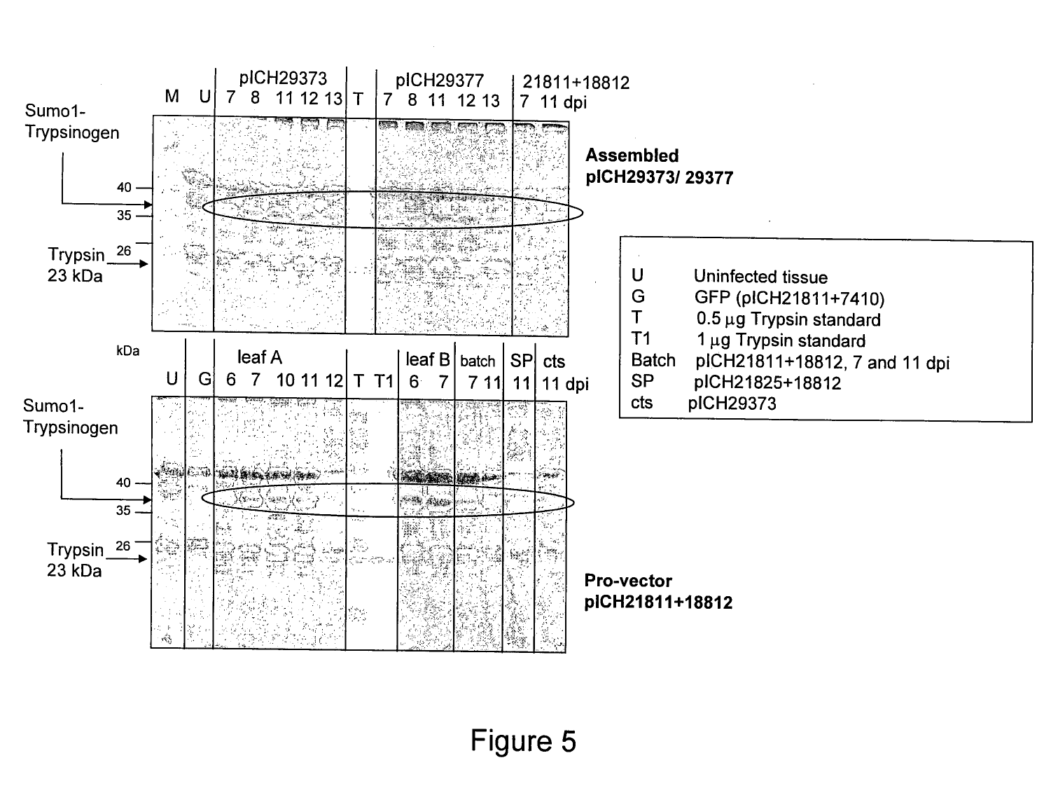

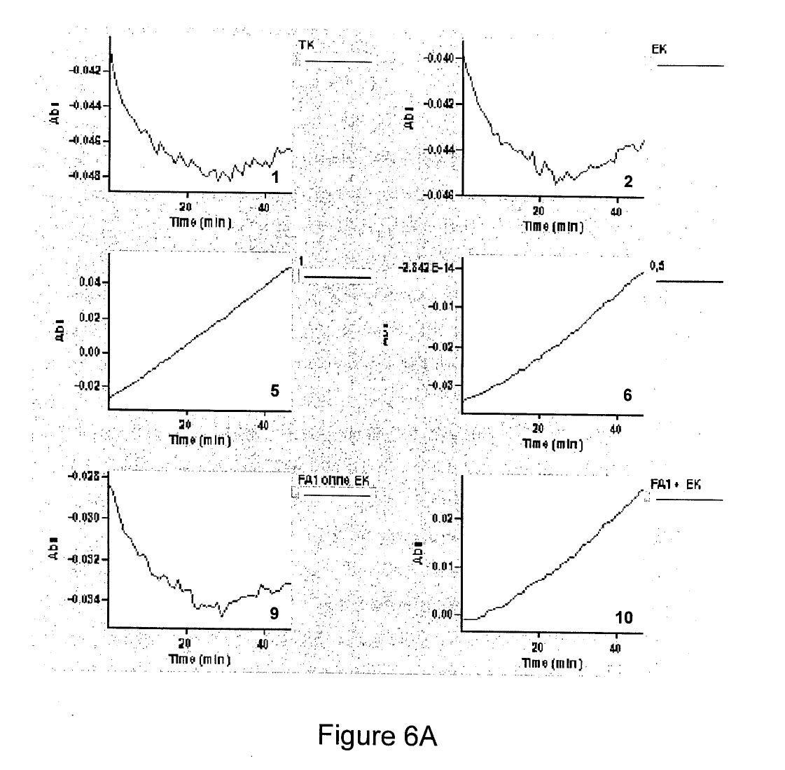

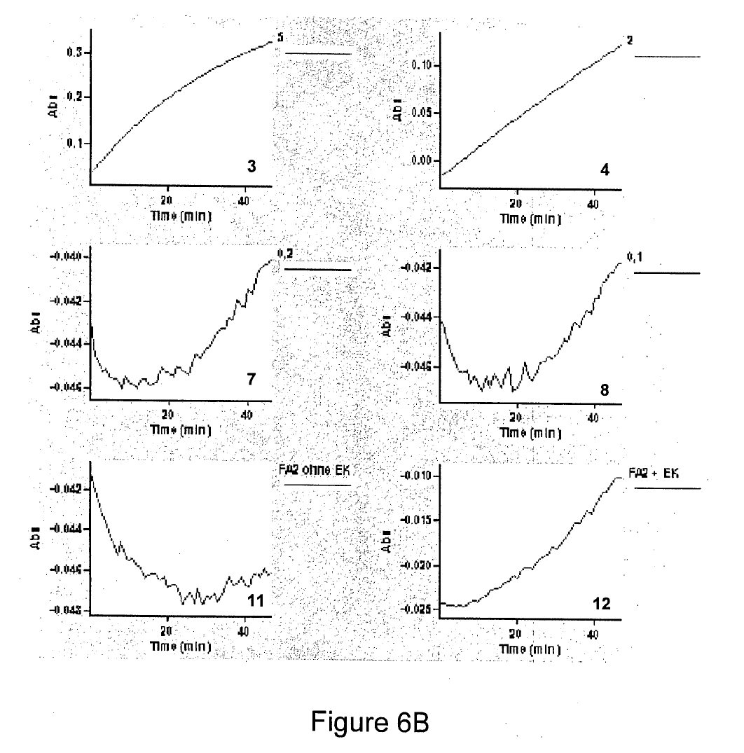

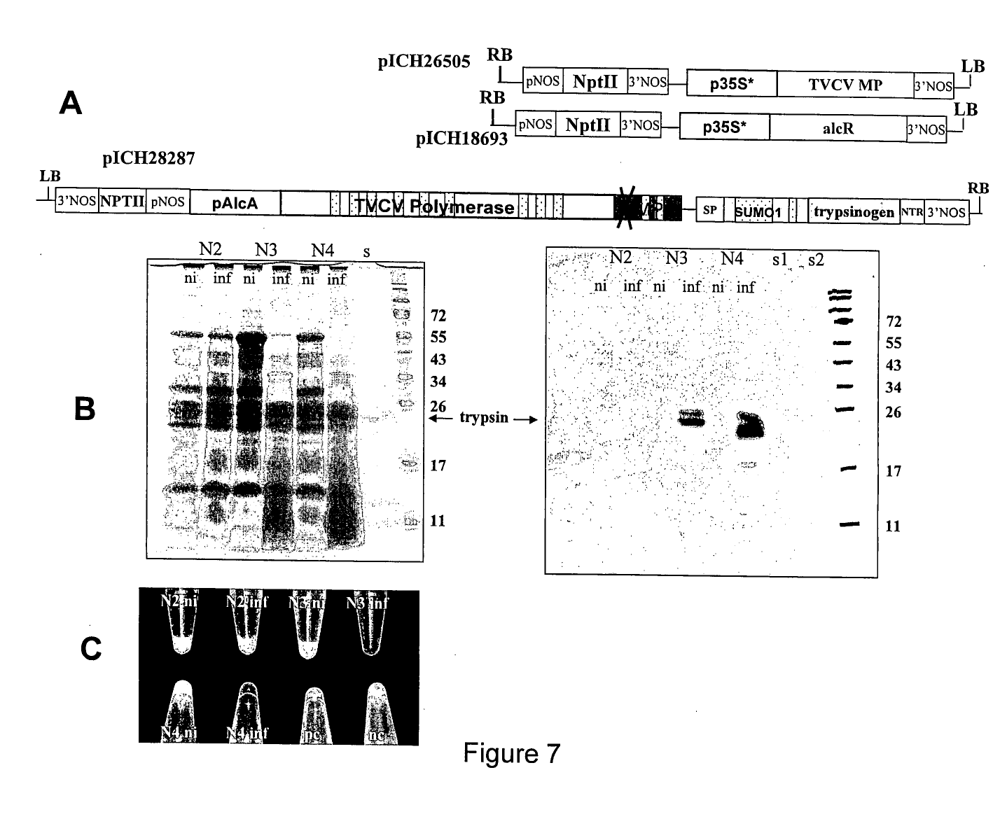

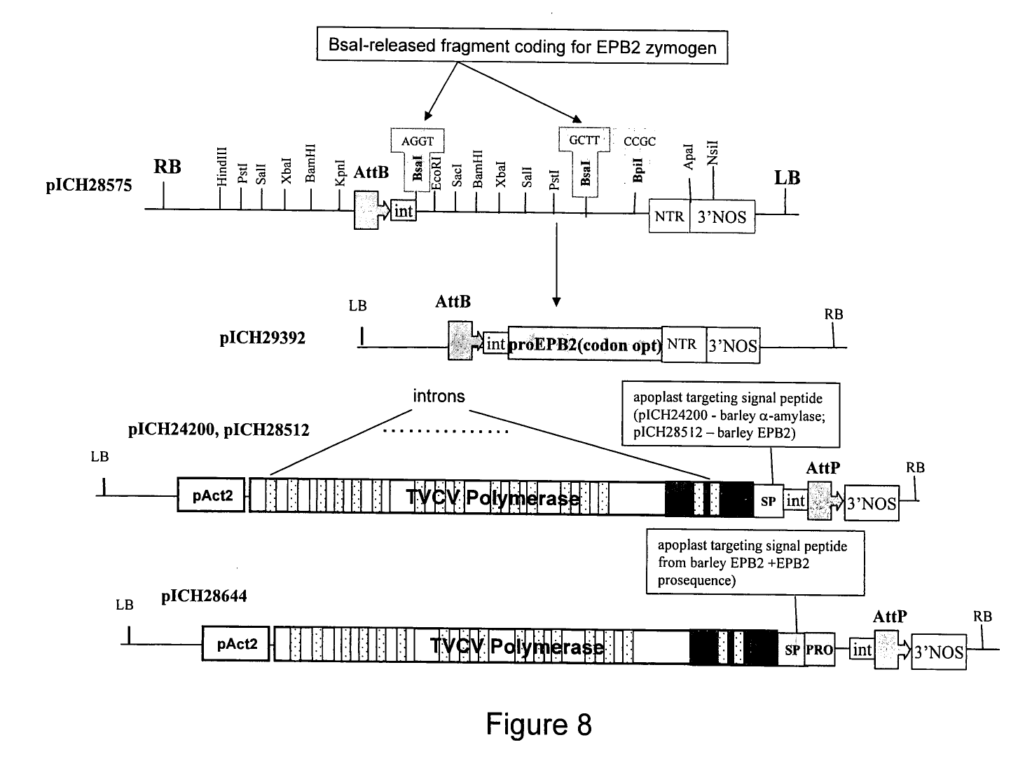

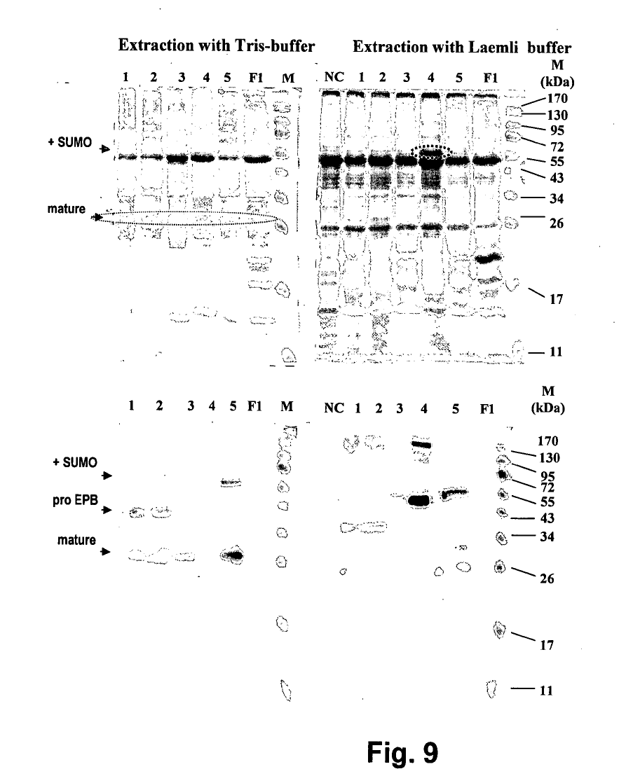

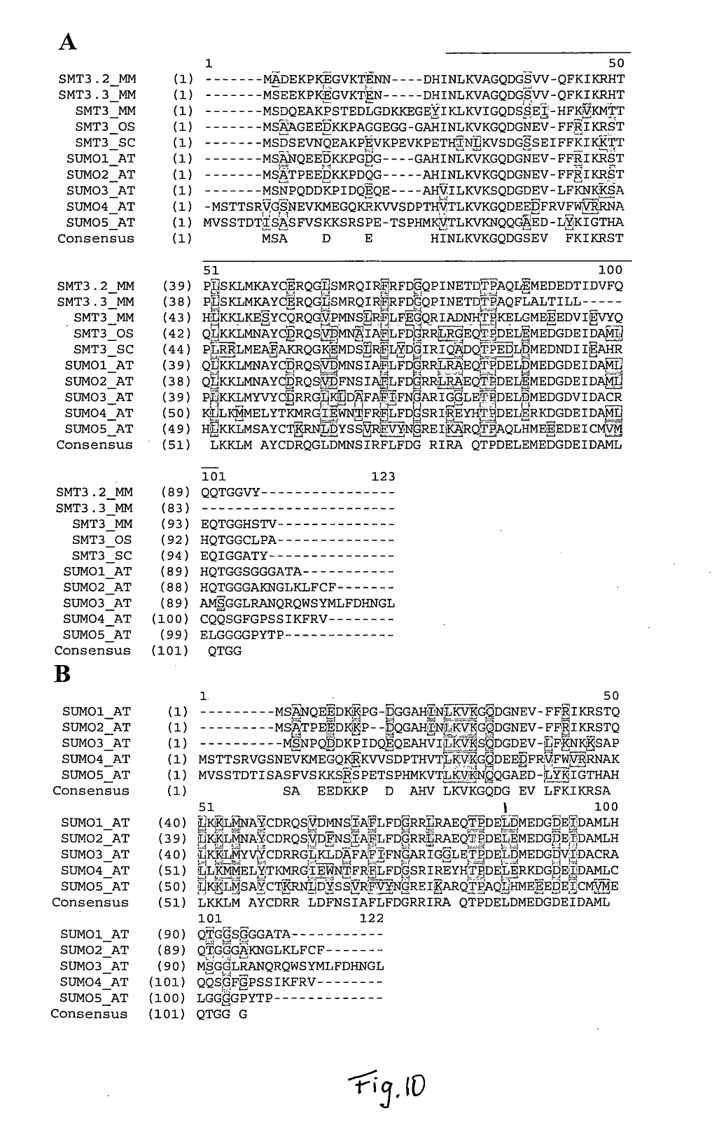

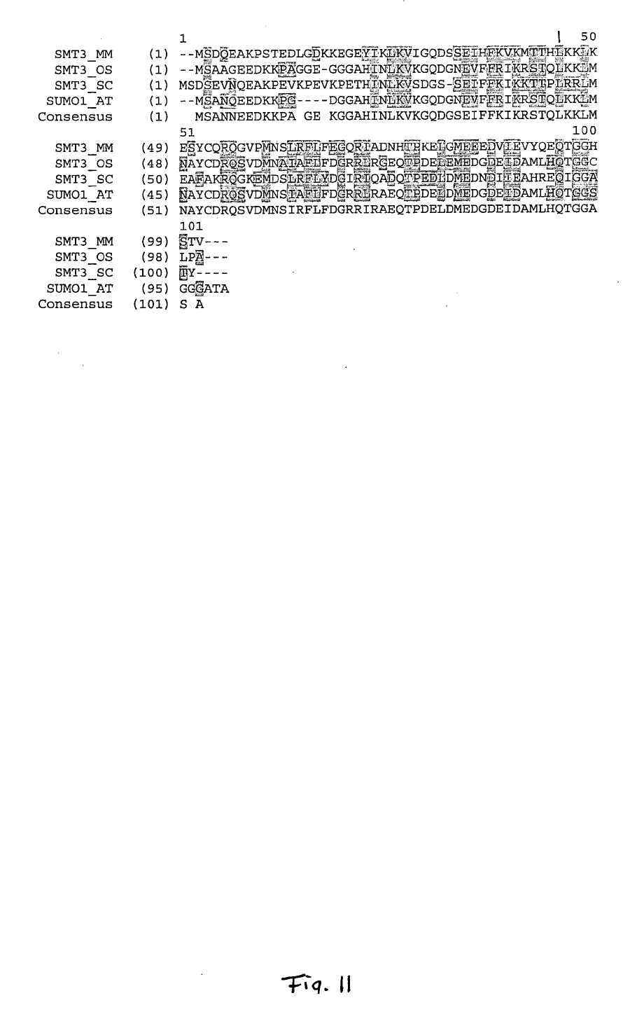

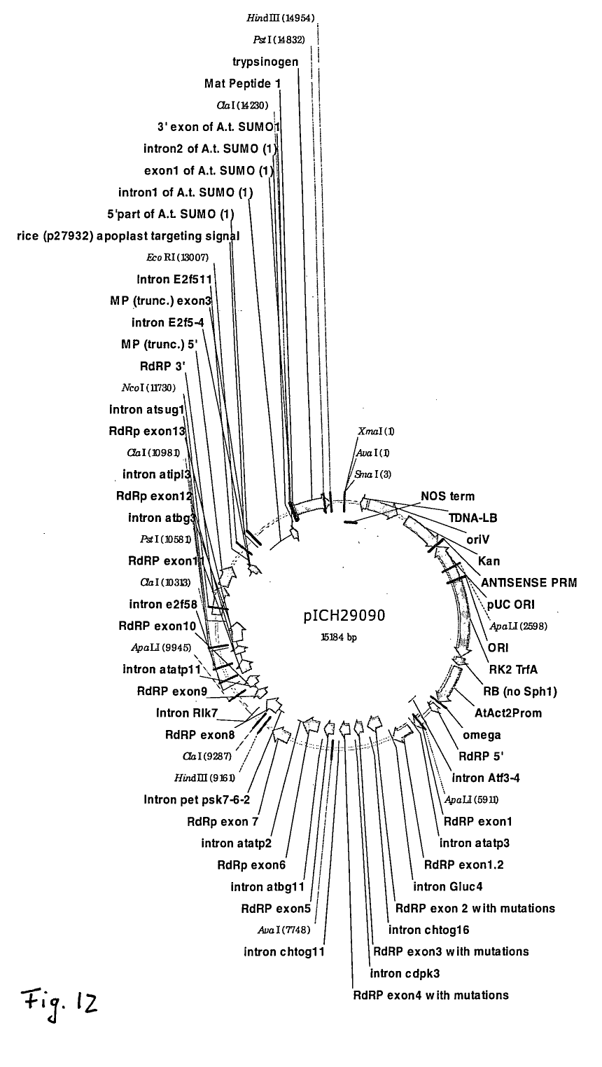

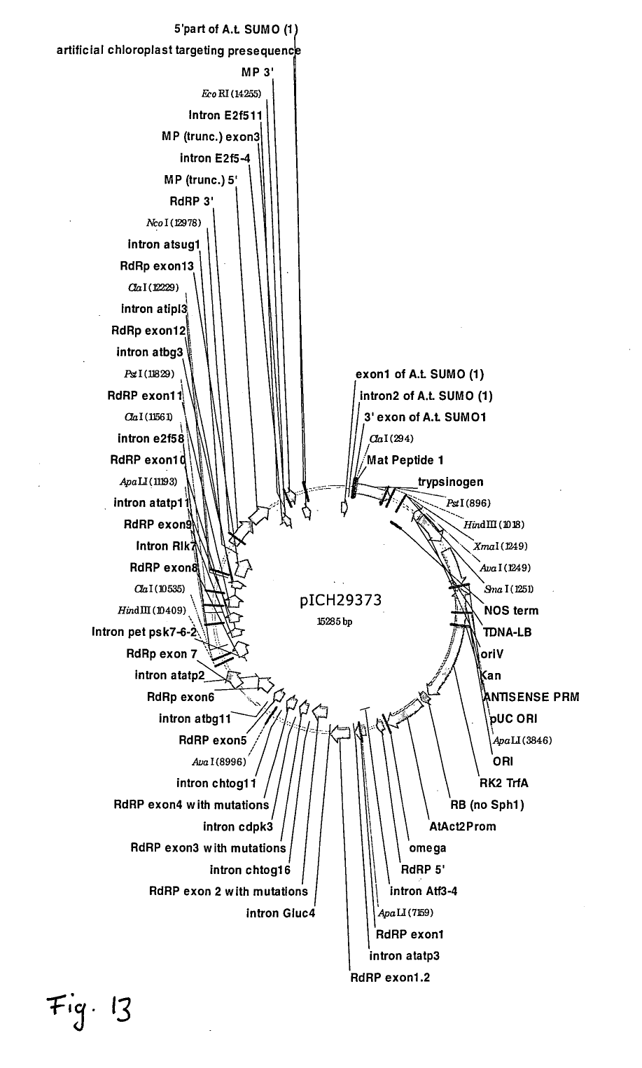

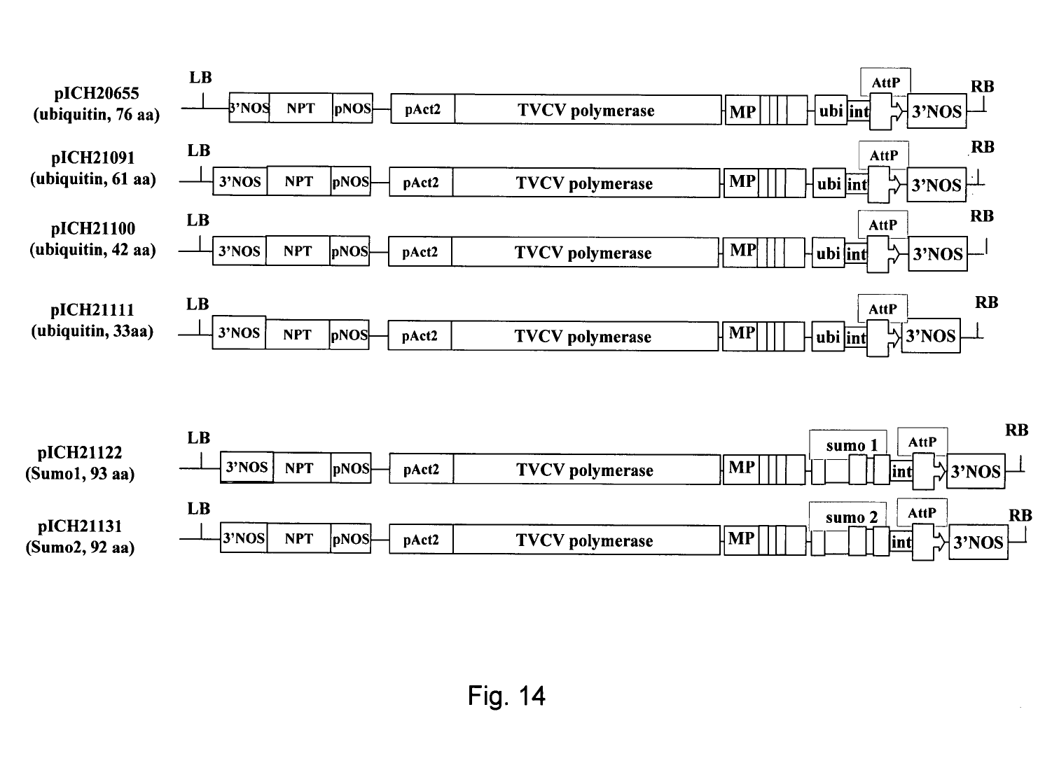

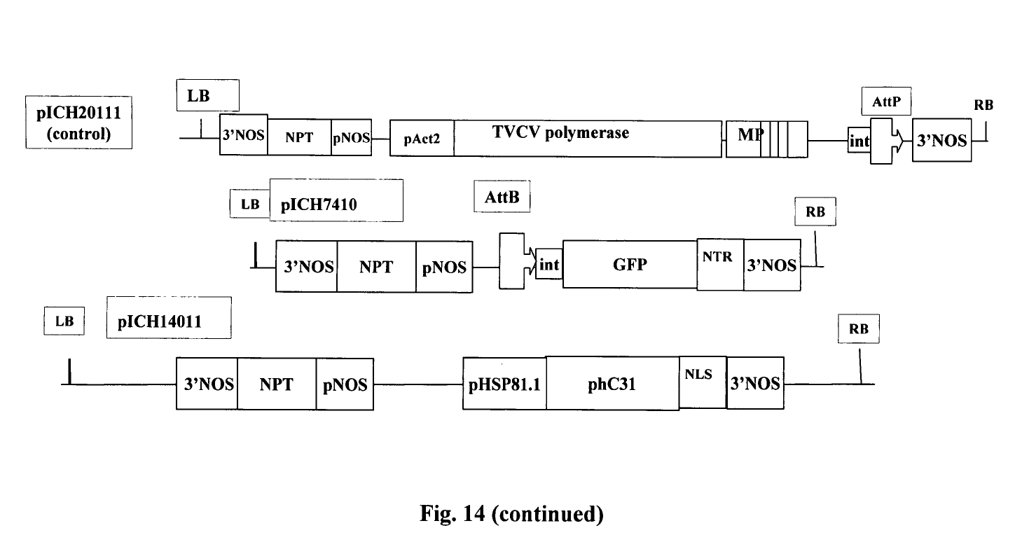

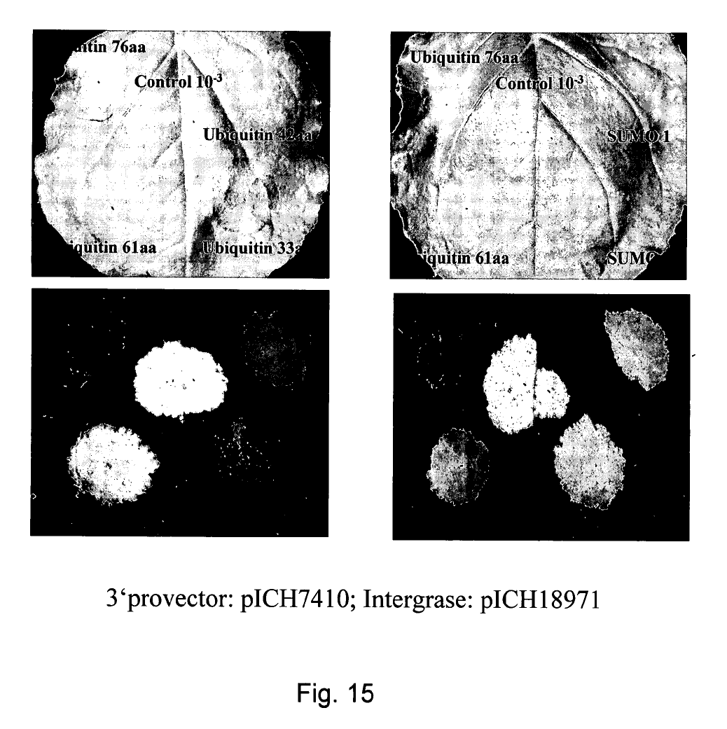

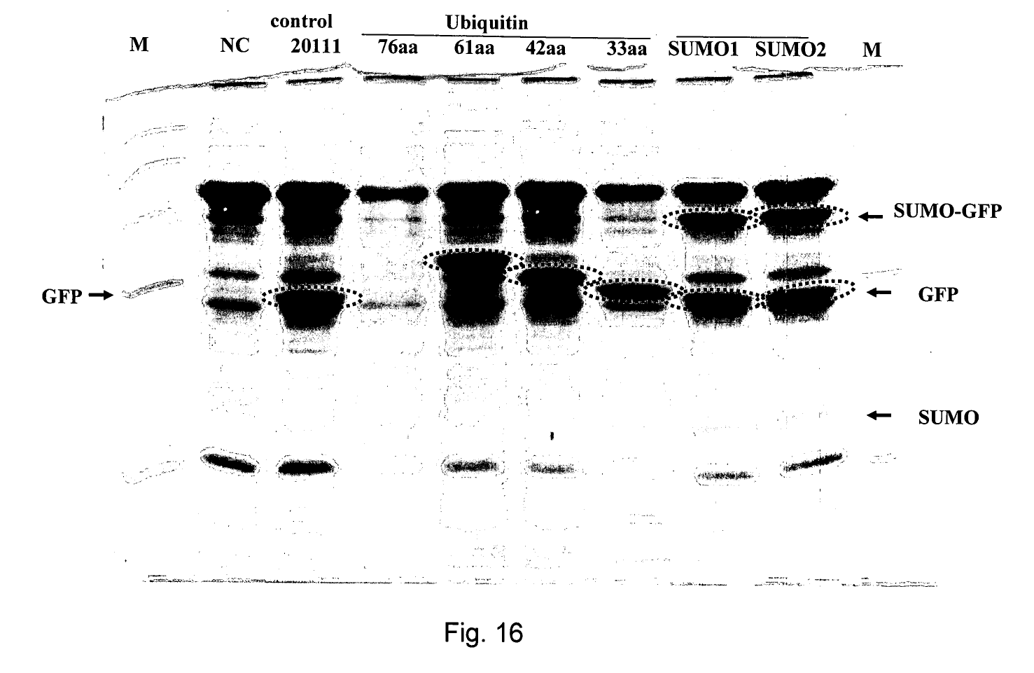

Targeting of trypsinogen into chloroplasts did not produce visible degradation of plant proteins, but resulted in a major coomassie-stained band on polyacryamide gel corresponding in size to a SUMO-trypsinogen fusion (FIG. 5). Accumulation of large amounts (ca. 2 mg/g of fresh leaf biomass) of said fusion in leaf chloroplasts is likely the result of formation of protein inclusion bodies, like in bacterial cells. Formation of inclusion bodies in chloroplasts is well known (Ketchner et al., 1995, Biol Chem., 270, 15299-15306; De Cosa et al., 2001, Nat Biotechnol., 19, 71-74; Fernandez-San Milán et al., 2003, Plant Biotechnol J., 1, 71-79; Fernandez-San Milán et al., 2007, J Biotechnol., 127, 593-604). The similarity to protein inclusion bodies from bacterial cells allows to use established technologies for solubilisation and refolding of bacterial protein inclusion bodies (for review and practical guide see: Singh S M, Panda A K. 2005, J Biosci Bioeng., 99, 303-310; Panda A K. 2003, Adv Biochem Eng Biotechnol., 85, 43-93; Cabrita L D, Bottomley S P. 2004, Biotechnol Annu Rev.; 10, 31-50; Mukhopadhyay A. 1997, Adv Biochem Eng Biotechnol., 56, 61-109; Mayer M, Buchner J. 2004, Methods Mol Med.; 94, 239-54; Misawa S, Kumagai I. 1999, Biopolymers., 51, 297-307). Indeed, in another embodiment of the invention (example 3), a successful approach for extraction and refolding of SUMO1-trypsinogen fusion is described by using slightly modified protocols for extraction of trypsinogen from bacterial inclusion bodies (Hohenblum et al., 2004, J. Biotechnol. 109, 3-11; Ahsan et al., 2005, Mol. Biotechnol., 30, 193-205; Kiraly et al., 2006, Protein Expr. Purif., 48, 104-111). Products of folding reaction were treated with enterokinase and then tested for the formation of enzymatically active trypsin using the kinetics of BAPNA cleavage. The results of these experiments are shown in FIG. 6. It is evident from presented data that refolded and enterokinase-treated SUMO1-trypsinogen fusion produces active trypsin capable of digesting the substrate BAPNA (see graphics 10 and 12 of FIG. 6). Formation of inactive inclusion bodies in plastids has the advantage that toxic effects on plastids by the protease are unlikely.

For large scale production of proteases in plants, a transgenic version of production host may be advantageous compared to transient expression system. Among transgenic vectors, those providing for controllable expression of the coding sequence of the invention are preferred. Controllable expression can help to further minimize cytotoxic effects of a protease. In case of stable integration of a vector expressing a SUMO-trypsinogen fusion into a plant genome, controllable vectors based on inducible expression of said vector is preferred. In the present invention, inducible promoters can be used to trigger production of a protease of interest in plants or plant cells. Inducible promoters can be divided into two categories according to their induction conditions: those inducible by abiotic factors (temperature, light, chemical substances) and those that can be induced by biotic factors, for example, pathogen or pest attack. Examples of the first category include, but are not limited, heat-inducible (US 05187287) and cold-inducible (US05847102) promoters, a copper-inducible system (Mett et al., 1993, Proc. Natl. Acad. Sci., 90 4567-4571), steroid-inducible systems (Aoyama & Chua, 1997, Plant J., 11, 605-612; McNellis et al., 1998, Plant J., 14, 247-257; US06063985), an ethanol-inducible system (Caddick et al., 1997, Nature Biotech., 16, 177-180; WO09321334; WO0109357; WO02064802), isopropyl beta-D-thiogalacto-pyranoside (IPTG)-inducible system (Wilde et al., 1992, EMBO J., 11:1251-1259) and a tetracycline-inducible system (Weinmann et al., 1994, Plant J., 5 559-569). One of the latest developments in the area of chemically inducible systems for plants is a chimaeric promoter that can be switched on by glucocorticoid dexamethasone and switched off by tetracycline (Bohner et al., 1999, Plant J., 19, 87-95). Chemically inducible systems are the most suitable for practicing the present invention. For a review on chemically inducible systems see: Zuo & Chua, (2000, Current Opin. Biotechnol., 11 146-151) and Moore et al., (2006, Plant J., 45: 651-683). It will be clear for the skilled person that any proteins required for the functionality of the chosen inducible system such as repressors or activators have to be expressed in said plant or said plant cells for rendering the inducible system functional. In one embodiment of the invention, ethanol inducible system for controlled release of viral replicon in plant cell is used. In example 4, an alcohol-inducible system described in detail in WO2007137788 was used for inducible expression of apoplast-targeted SUMO1-trypsinogen fusion. The results obtained demonstrate that tightly controlled inducible expression of protease from plant viral vector is obtained. FIG. 7 (B, C) shows expression of enzymatically active trypsin in different transgenic plants under inducible conditions.

In example 5 of this invention, we present data of SUMO-EPB2 protease precursor expression in plant cells. It is evident (FIG. 9, upper right panel, line 4) that a very high expression level of SUMO-EPB2 protease precursor fusion targeted into chloroplasts was achieved. Like in the case with chloroplast-targeted SUMO-trypsinogen fusion, SUMO-EPB2 precursor fusion accumulates in chloroplasts in the form of inclusion bodies that require strong denaturing buffers for their extraction from plant tissue. The extracted protein can be refolded in a way similar to the one described for chloroplast-targeted trypsinogen fusion with SUMO.

Considering that protease protein production in this invention includes the fusion of the protease with a signaling or transit peptide and SUMO, the separation of the protein of interest from the fusion protein shall be considered. In the invention, the use of SUMO and a protease precursor in such fusions introduces at least two cleavage sites. One cleavage site is located between the C-terminus of SUMO protein and the N-terminus of the protease precursor. This cleavage site is recognized by SUMO-specific proteases. Therefore, separation of the protease from SUMO is not an issue. Plant cells like all other eukaryotes, contain potent SUMO proteases that cleave proteins at the end of SUMO, thus precisely removing from SUMO any C-terminal extensions and fusions (Kurepa et al., 2003, J. Biol. Chem., 278, 6862-6872; Colby et al., 2006, Plant Physiol., 142, 318-332; Novatchkova et al., 2004, Planta, 220, 1-8; Hay, R T., 2004, Trends Cell Biol., 17, 370-376; Johnson et al., 2004, Annu Rev Biochem., 73, 355-382). In the invention, the protease precursor or zymogen is used for fusion with SUMO protein. The protease precursor contains yet another cleavage site not far from the N-terminus of said precursor. Cleavage at this cleavage of said site is important for the maturation of the precursor into the active protease. In case of trypsinogen, precursor of trypsin, enterokinase-mediated removal of a hexapeptide from the N-terminal end of trypsinogen produces trypsin. From our results with apoplast targeted SUMO-trypsinogen fusion, it is evident that a protease with enterokinase-like activity capable of a cleaving hexapeptide from trypsinogen must be present in plants. If desired, a protease capable or producing the protease produced according to the invention from its zymogen may be applied. Notably, such treatment step will be used during isolation and/or purification of the protease of interest that was targeted to plastids.

The presence of a cleavage site within the zymogen allows removal of any fusion protein linked to the N-terminus of said zymogen. Therefore, the use of intact C-terminus of SUMO protein in order to provide cleavage with a SUMO-specific proteases is not necessary in the invention. This allows to use a truncated version of SUMO protein in the fusion protein of the invention and other derivatives of SUMO protein.

### EXAMPLES

### Example 1

### Cloning of Bovine Coding Sequences Encoding for Trypsinogen and Trypsin and Their Integration Into Plant Viral Vectors

Commercially available calf thymus genomic DNA (Sigma-Aldrich., cat. No. D4764) was used as the template for cloning coding regions for trypsinogen and trypsin proteins. The partial coding sequence for activation peptide and pancreas cationic trypsinogen (Core Nucleotide database Acc. No D38507, also see FIG. 1 (A) was used for primers design. Nine primers containing BsaI restriction sites were synthesized in order to amplify exon sequences encoding bovine cationic trypsin and trypsinogen proteins. The primer sequences and general scheme of cloning strategy are shown in FIG. 2. The A-tailed PCR products were subcloned into pGemT T-tailed vector (Promega, Cloning kit Cat. No. A3600). Nine primers were designed in order to amplify the coding sequences from genomic DNA and avoid introns. The primers also introduced flanking BsaI sites into PCR fragments. Five different fragments covering cDNA coding region for trrypsinogen/trypsin were independently subcloned in pGemT vectors and sequenced. Clones with correct sequences were used for assembly of coding sequences for trypsinogen or trypsin proteins by subcloning appropriate PCR clones as BsaI fragments into BsaI and HindIII-digested vector pICH10990 yielding vectors pICH18812 (trypsinogen, FIG. 2) and pICH18820 (trypsin, FIG. 2). Use of BsaI restriction sites provides the universal approach to create any desired compatible sticky ends flanking digested DNA fragments and thus allows to perform correct assembly of several DNA fragments in one cloning step. The vectors pICH18812 and pICH18820 were further used as 3′ provectors in site-specific recombination-mediated assembly of DNA encoding for complete viral vector. The principle of such DNA modules assembly in planta is described by Marillonnet et al., 2004, Proc. Natl. Acad. Sci. USA, 101, 6852-6857. The approach allows high throughput testing of different targeting signals and fusion proteins in combination with the protein of interest for optimizing said protein expression level.

The Arabidopsis thaliana SUMO1 gene (gene ID 828791) was cloned using genomic DNA as template for PCR amplification. The PCR product containing two original SUMO1 introns, was cloned into intermediate vectors using standard molecular biology techniques ((Sambrook, Fritsch & Maniatis, 1989, Molecular Cloning: a Laboratory Manual, CSH, NY), and used in constructs design.

The viral vector modules with signal peptide, transit peptide-SUMO1 and signal peptide-SUMO1 fusions encoding for 5′-viral pro-vectors and assembled viral vectors were designed using cloning approaches, as it described by Marillonnet et al., 2005, Nat. Biotechnol., 23, 718-723. The restriction maps of whole plasmids and complete coding sequences for the T-DNA regions of assembled viral vectors pICH29090 and pICH29373 (FIG. 3) are shown FIGS. 12 and 13, respectively.

The complete sequence of pICH29090 is shown in SEQ ID NO: 1. The sequence encoding the apoplast targeting signal peptide of pICH29090 is shown in SEQ ID NO: 2. The sequence encoding the apoplast targeting signal peptide of pICH29373 is shown in SEQ ID NO: 3. The sequence of pICH29373 is identical to that of pICH29090 except that the sequence encoding the apoplast targeting peptide of pICH29090 is replaced by the sequence encoding the plastid transit peptide shown in SEQ ID NO: 3.

### Example 2

Transient Expression of Bovine Trypsinogen in N. benthamiana Using Plant Viral Vectors

### Agroinfiltration

All constructs described in example 1 were electroporated into Agrobacterium tumefaciens GV3101. Agroinfiltrations of N. benthamiana plants were done essentially as described in Marillonnet et al., 2004, Proc Natl Acad Sci USA, 101:6852-6857. In case of provectors, three agrobacterial strains containing 5′ provector encoding for targeting signal peptide and SUMO1 fusion or any of the fusion/targeting sequence (not shown), 3′ provector encoding the trypsinogen or trypsin genes and a source of a site-specific recombinase (pICH14011, FIG. 3) for assembly of viral pro-vectors in planta via site-specific recombination to viral vector were mixed together and used for infiltration. Small-scale infiltrations were done with a syringe; large-scale infiltrations were done using a vacuum device (Marillonnet et al., 2005, Nat Biotechnol., 23:718-723). Agrobacterial strains containing assembled viral vectors (pICH29090, pICH29373, FIG. 3) were agro-infiltrated independently.

Analysis of SUMO1-Trypsinogen Fusion Expressed in N. benthamiana Leaves

All recombinant protein fusions were extracted from infiltrated N. benthamiana leaves 7-12 days after infiltration and analysed by electrophoretic separation in polyacrylamide gels as previously described (Marillonnet et al., 2004, Proc Natl Acad Sci USA, 101:6852-6857; Marillonnet et al., 2005, Nat Biotechnol., 23:718-723). Plant leaf tissue was harvested from different leaves of young (y) or old plants. Tissue was extracted with 3 volumes of 0.15 M Tris-HCl pH8.1; 2mM EDTA, incubated for 10 minutes on ice and centrifuged for 12 minutes at 13 Krpm, 4° C. Supernatant was mixed with equal volume of 2× Laemmli buffer (125 mM Tris/HCl pH 6.8, 10% mercaptoethanol, 20% glycerol, 0.01% bromphenole blue, 4% SDS) and 0.004 ml of mixture corresponding to 1.3 mg of starting leaf tissue was loaded on gels.

The results of electrophoretic analysis are shown in FIG. 4 (A). The position of trypsin bands was identified by using Western blotting (FIG. 4-B) with anti-bovine trypsinogen polyclonal rabbit antibodies (Rockland/Biomol GmbH. Hamburg, cat No. 100-4180). It corresponded to clearly visible 23 KDa coomassie-stained band on polyacrilamyde gel. About 0.14 mg trypsin per gram of fresh leaf biomass were expressed in leaf tissue at 7 days post infection in a typical experiment.

### Measurement of Plant-Made Recombinant Trypsin Activity

The relative activity of plant-expressed trypsin in comparison with commercially available trypsin samples (ICN Biomedicals Inc., cat. 101789, 10 mg/ml in 1 mM HCl, working solution 1:10 to 1:100 diluted in 1 mM HCL) was measured by using milk assay. The assay was performed as follows:

0.005-0.02 ml of plant extract in 0.15 M Tris-HCl pH 8.1; 2 mM EDTA 8,0 buffer is mixed with 0.02 ml of 3% solution of dry milk powder (Roth, Cat. No. T145.2) in TBS (25 mM tris-HCl, 142 mM NaCl) and incubated at room temperature until positive control sample with commercially available trypsin does not start to clarify the milk solution due to the digestion of milk protein. Clear solution in test samples means the presence of active trypsin. If the solution remains milky, no trypsin activity is present. The results of the test are shown in FIG. 4-C.

### Analysis of Chloroplast-Targeted SUMO1-Trypsinogen Fusion

Agroinfiltration of plants with assembled viral vector and provectors providing for expression of chloroplast-targeted SUMO1-trypsinogen fusion was performed as described above for apoplast-targeted SUMO1-trypsinogen fusion. The results of electrophoretic analysis of the expression level are shown in FIG. 5 (coomassie-stained bands corresponding to recombinant protein fusion are circled). It is evident that a large amount (ca. 2 mg/g of fresh leaf biomass) of inactive (no degradation of protein in coomassie-stained gel was detected) SUMO1-trypsinogen fusion accumulated in plant tissue.

### Example 3

### Extraction and Reactivation of Chloroplast Targeted SUMO1-Trypsinogen Fusion

Extraction of SUMO1-Trypsinogen Fusion from Chloroplasts

The protein fusion accumulated in chloroplasts can be extracted with SDS-PAGE sample (Laemmli) buffer at 95° C. (see example 2) and at least partially with buffers containing chaotropes. The current extraction method was established based on methods suitable for solubilisation of inclusion bodies (IBs) occurring in many recombinant protein expressions in E. coli including trypsinogen (Buswell et al., 2002, Biotechnol. Bioeng., 77, 435-444; Hohenblum et al., 2004, J. Biotechnol., 109, 3-11; Ahsan et al., 2005, Mol. Biotechnol., 30, 193-205; Király et al., 2006, Protein Expr. Purif., 48, 104-111). 5.5 g of plant leaf material containing chloroplast targeted SUMO1-trypsinogen fusion was sequentially treated with the set of following extraction buffers:
E1: 100 mM Tris-HCl, 200 mM NaCl, 1 mM EDTA, pH 8.5
E2: 60 mM EDTA, 2% Triton-100, 1.5 M NaCl, pH 8.8
E3: 4 M urea, acetic acid, pH 4.0
E4: 6 M GuaHCl, 100 mM Tris-HCl, 1 mM EDTA, 100 mM DTT, pH 8.8

Extraction 1: 30 ml buffer E1 was added to the leaf material. The suspension was thoroughly mixed using an ultraturrax (6-7 times 30 s), and kept on ice between the 3 mixing steps. Overall mixing time 10 min. The suspension was centrifuged 15 min at 40000g and 4° C.

Extraction 2: 25 ml buffer E2 were added to the pellet of extraction step 1. The suspension was thoroughly mixed using an ultraturrax (3 times 1 min), and kept on ice between the mixing steps. Overall mixing time 10 min. The suspension was centrifuged 15 min at 40000 g and 4° C.

Extraction 3: 25 ml buffer E3 were added to the pellet of extraction step 2. The suspension was thoroughly mixed using an ultraturrax (3 times 1 min) and kept on ice between the mixing steps. Overall mixing time 10 min. The suspension was centrifuged 15 min at 40000 g and 4° C. This washing step was repeated 3 times.

Extraction 4: 10 ml buffer E4 was added to the pellet of extraction step 3. The suspension was thoroughly mixed using an ultraturrax (5 min), and incubated further for 1 h on a rolling mixer at room temperature. The suspension was centrifuged 15 min at 40000 g and 4° C. The supernatant, assumed to contain solublised SUMO-trypsinogen, was applied to a HiPrep Desalting column to exchange the buffer to 8 M urea, acetic acid, pH 4.0. The fractions containing protein were pooled. All fractions were analysed by SDS-PAGE and Westernblot (not shown).

The final extraction of unsoluble proteins is achieved with the GuaHCl containing buffer. In preliminary experiments, this buffer was found to be suitable to extract more SUMO1-trypsinogen compared to a similar buffer containing 9 M urea as chaotrope.

Additional purification of extracted SUMO1-trypsinogen was carried out using Q Hyper D 20 anion exchange chromatography (Pall, Biosepra, code no. 200683, column 4.6×100 mm). The sample was prepared by adding 0.5 volume of 200 mM Tris-HCl, 1 mM EDTA, pH 8.5, to one volume of solubilised SUMO1-trypsinogen from extraction 4. The pH was adjusted to >8.0 by addition of 1 M NaOH and final solution was loaded onto the column. The elution was carried out by linear NaCl gradient (starting buffer: 50 mM Tris-HCl, 8 M urea, pH 8.5; final buffer: 50 mM Tris-HCl, 8 M urea, 1 M NaCl, pH 8.5; flow rate: 1 ml/min; 20 column volumes, elution fraction volume: 0.5 ml. 8 M urea was used to keep SUMO-trypsinogen fusion solubilised. The resulting purity of SUMO-trypsinogen was comparable to typical proteins solubilised from bacterial inclusion bodies. The final pool contained an overall amount of 0.6 mg in 3 ml.

### SUMO1-Trypsinogen Folding and Cleavage of Fusion to Produce Active Trypsin

The folding of purified SUMO1-trypsinogen fusion was carried out as described by Hohenblum and colleagues (J. Biotechnol., 109 (2004) 3-11) with some modifications. Concentration of the purified extract of SUMO1-trypsinogen to 150 μl was carried out by using Vivaspin 500 (Sartorius, MWCO 3,000; prod no. VS0191; Lot no. 07VS50030). Recovery from the Vivaspin concentrator included rinsing with 8 M urea, 50 mM Tris, pH 8.6. Final volume of the concentrated samples 700 μl. To the concentrated sample, 1 M DTE was added to a final concentration of 10 mM and the solution was incubated for 2.5 hours at 37° C. Then, 1 volume of 200 mM GSSG (oxidised glutathion), 8 M urea, pH 8.6 was added and the mixture was incubated for 3 hours at 37° C. After incubation, the buffer was replaced to 50 mM Tris-HCl, 8 M urea, pH 8.6 by using HiTrap desalting columns (GE Healthcare, cat. no. 17-1408-01). Then dilution of the solubilisates into folding buffer (two different buffers, 1:20 dilution; folding buffer 1: 50 mM Tris-HCl, 50 mM CaCl2, 3 mM GSH, 0.3 mM GSSG, pH 8.6; folding buffer 2: 50 mM Tris-HCl, 50 mM CaCl2, 700 mM Arg, 3 mM GSH) and incubation of the folding reaction was carried out at 4° C. for at least 16 hours. After incubation, the folding reaction was concentrated 10-fold with Vivaspin 20 concentrators (Sartorius, MWCO 5,000; prod. no. VS2011, Lot 06VS2050). Then, the folding buffer was replaced by cleavage buffer (20 mM Tris-HCl, 50 mM NaCl, 2 mM CaCl2, pH 8.0) using HiTrap desalting column. The resulting samples were analysed by SDS-PAGE and activity assays were made (results are not shown).

In order to test the quality of SUMO1-trypsinogen folding, enterokinase (stock solution: 1 mg/ml of enterokinase (Sigma, cat. no. E0885) in cleavage buffer) was added to the folding samples. In case of a successful folding, enterokinase cleavage should lead to the formation of active trypsin that can be detected due to the cleavage of the chromogenic substrate BAPNA (Sigma, cat. no. B4875). The enterokinase cleavage and the analytical methods were established with commercially available trypsinogen. In accordance with the SUMO1-trypsinogen concentration in the folding reaction, the analytical method was established with trypsinogen concentrations between 0.1 and 50 μg/ml. The BAPNA assay system was optimised to detect very small amounts of trypsin in enterokinase treated folding reactions. The BAPNA assay was performed as follows:

Ten microliters of enterokinase were added to 990 pl folding sample in cleavage buffer and the mixture was incubated at 37° C. for >16 hours. The sample was pipetted into a cuvette and 50 μl of 2 mM BAPNA solution in cleavage buffer were added. The BAPNA cleavage was detected at 37° C. by absorption spectroscopy at 405 nm over a time period of 60 min. The results (kinetic of absorption) were evaluated in comparison with results obtained from control samples obtained from trypsinogen standard. The results of cleavage experiments are shown in FIG. 6.

### Example 4

Use of Alcohol-Inducible System for the Expression of Apoplast Targeted SUMO-Trypsinogen Fusion in Transgenic N. benthamiana Plants

### Constructs Design

The constructs for inducible expression of trypsin in transgenic plants are shown in FIG. 7A. Plasmid pICH28287 is very similar to the plasmid coding for assembled viral vector pICH29090 (FIG. 3) except that the promoter of Arabidopsis actin 2 gene was replaced with ethanol inducible alcA promoter and a frameshift mutation was introduced into the coding sequence of MP. Description of the ethanol-inducible system for expression of recombinant proteins in plants using standard transcriptional vectors was provided in detail in several publications (Caddick et al., 1997, Nature Biotech., 16 177-180; WO09321334; WO0109357; WO02064802). Ethanol-inducible system used in this invention for controlling plant viral vector-based expression was described in detail in our PCT application WO 2007/137788.

N. benthamiana plants were transformed with pICH28287 according to standard protocols (Horsh et al., 1985, Science, 227 1229-1231). Regenerated plants were analysed for the presence of the transgene by agroinfiltration with the constructs providing for alcR transcriptional activator and functional MP (pICH18693 and pICH26505, respectively, see FIG. 7-A) followed by ethanol treatment. Analysis of three (N2, N3, N4) such transgenic plants for the presence of trypsin enzymatic activity is shown in FIG. 7-B, C. Preparation of samples, gel-electorphoresis, Western blotting and milk assay were performed as described in example 2. Clearly, two out of three plants express trypsinogen upon induction. Activity is shown both in the milk assay and by degradation of proteins in the Coomassie-stained gel.

### Example 5

Cloning of Barley (Hordeum vulgare) Coding Sequences Encoding for Cysteine Endoprotease B (EPB2) Precursor and its Integration into Plant Viral Vectors

The codon-optimised sequence of barley cysteine endoprotease B isoform 2 (EPB2) gene (GeneBank Acc. No. U19384) was custom-synthesized (GENEART AG, Regensburg, Germany). The sequence of the gene and its translation product are shown in FIG. 1(B). As a matter of convenience, the sequence was flanked with two BsaI sites that were used for recloning of the gene into BsaI digested provector pICH28575, yielding provector pICH29392. Schematic representations of provectors and cloning procedures are shown in FIG. 8. The vector pICH29392 was further used as 3′ provectors in site-specific recombination-mediated assembly of DNA encoding for complete viral vector. The principle of the assembly of such DNA modules in planta is described by Marillonnet et al., 2004, Proc. Natl. Acad. Sci. USA, 101, 6852-6857. This approach allows high-throughput testing of different targeting signals and fusion proteins in combination with the protein of interest for optimizing the expression level of said protein. The 5′ provectors used in combination with pICH29392 are pICH24200, pICH28512, pICH28644 (FIG. 8) and pICH21811, pICH21825 (FIG. 3).

Analysis of SUMO1-EPB2 Zymogen Fusion Expressed in N. benthamiana Leaves

All recombinant protein fusions were extracted from infiltrated N. benthamiana leaves 7-12 days after infiltration and analysed by electrophoretic separation in polyacrylamide gels as previously described (Marillonnet et al., 2004, Proc Natl Acad Sci USA, 101:6852-6857; Marillonnet et al., 2005, Nat Biotechnol., 23:718-723). It was found that the expression level reached a maximum 8 days after infiltration.

Plant leaf tissue was extracted with 5 volumes of tris extraction buffer (0.1 M Tris-HCl pH 8.0; 5 mM EDTA, 2 mM mercaptoethanol, 0.1% SDS, 15% glycerol), incubated for 10 minutes on ice and centrifuged for 12 minutes at 13 Krpm, 4° C. The supernatant was mixed with equal volume of 2× Laemmli buffer (125 mM Tris/HCl pH 6.8, 10% mercaptoethanol, 20% glycerol, 0.01% Bromphenole blue, 4% SDS) and incubated in a boiling water bath before loading on gels. Alternatively, plant leaf tissue was extracted with 10 volumes (w/v) of 1× Laemmli buffer (62.5 mM Tris/HCl pH 6.8, 5% mercaptoethanol, 10% glycerol, 0.005% Bromphenole blue, 2% SDS). Extracts were incubated in a boiling water bath before loading on gels.

The results of electrophoretic analysis are shown in FIG. 9. The position of mature EPB2 (25 kDa), EPB2 propeptide (38 kDa) and SUMO1-EPB2 propeptide fusion are shown by arrows. The positions of EPB2-containing bands was confirmed by using Western blotting (FIG. 9, lower panel) with anti-EPB2 polyclonal rabbit antibodies.

### Example 6

### Expression of GFP Fusions With SUMO and Ubiquitination

GFP fusions with full-length A. thaliana ubiquitin as well as its N-terminally truncated versions were tested using plant virus-derived expression system. The constructs used in the experiment are shown in FIG. 14. Different combinations of 5′-provectors encoding for different fusion proteins were tested in combination with 3′-provector encoding for GFP. The constructs were assembled in planta at presence of integrase phiC31, as it described earlier (Marillonnet et al., 2004, Proc Natl Acad Sci USA. 101:6852-6857). In the same experiment, we also tested two different A. thaliana SUMO fusions (SUMO1 and SUMO2).

The pictures of infiltrated N. benthamiana leaves under day light and UV light (to monitor for GFP expression) are shown in FIG. 15. It is evident that GFP fusion with full-length ubiquitin (ubiquitin 76 aa) and one of its truncated versions (ubiquitin 33 aa) has cytotoxic effect on the leaf tissue. Fusion of GFP with two other truncated versions of ubiquitin (ubiquitin 61 aa; ubiquitin 42 aa) did not show noticeable cytotoxic effect, but N-terminal deletion of ubiquitin (15 and 43 aa, respectively) compromised cleavage of ubiquitin derivative from fusion product (FIG. 16).

SUMO-GFP fusions did not show cytotoxic effect (FIG. 15), were expressed at high level and SUMO was cleaved off in planta from GFP to a large extent (FIG. 16).

The entire disclosure of European patent application No. 08 004 005.8 filed on Mar. 4, 2008 including description, claims and figures is incorporated herein by reference.

1. A process of producing a protease in a plant or in plant cells, comprising
(a) providing a plant comprising a heterologous nucleotide sequence comprising a coding sequence encoding a fusion protein, said fusion protein comprising in the following order (i) to (iii) in N-terminal to C-terminal direction:
(i) an apoplast or plastid signal peptide;
(ii) a SUMO protein or a derivative of a SUMO protein; and
(iii) a zymogen of said protease, and
(b) expressing said fusion protein.

2. The process according to claim 1, wherein said plant or said plant cells provided in step (a) is/are stably transformed on a nuclear chromosome with said nucleotide sequence comprising said coding sequence.

3. The process according to claim 1, wherein said nucleotide sequence encodes a replicon comprising said coding sequence or a transcript of said coding sequence.

4. The process according to claim 3, wherein said replicon is an RNA viral replicon.

5. The process according to claim 1, wherein said nucleotide sequence comprises a promoter upstream of said coding sequence, said promoter allowing expression of said coding sequence in vegetative tissue of said plant.

6. The process according to claim 5, wherein said promoter is an inducible promoter.

7. A process of producing a protease in a plant or in plant cells, comprising providing a plant with a replicon comprising a coding sequence encoding a fusion protein, said fusion protein comprising in the following order (i) to (iii) in N-terminal to C-terminal direction:
(i) an apoplast or plastid signal peptide;
(ii) a SUMO protein or a derivative of a SUMO protein; and
(iii) a zymogen of said protease.

8. The process according to claim 7, where said replicon is a plant viral expression vector.

9. The process according to claim 7, wherein said replicon is an RNA replicon, and said plant is provided with said RNA replicon by transforming said plant or said plant cells with a DNA vector encoding said RNA replicon or with two or more DNA vectors encoding together said RNA replicon.

10. The process according to claim 1, wherein said derivative of a SUMO protein is capable of increasing the expression level of said protease or of a protein comprising said protease compared to the absence of said derivative.

11. The process according to claim 1, wherein said derivative of a SUMO protein comprises an amino acid sequence segment of at least 50 contiguous amino acid residues, said segment comprising any one of the following amino acid consensus sequences:
-L/F/M-X₁₉-F/I-X₃-G/D-X₇-T/S-P/A-;
-L/F/M-X₁₉-F/I-X₃-G/D-X₇-T-P-;
-L-(X)₁₉-F-X₃-G-X₇-T-P-;
-L-X₁₉-F-X₃-G-X₇-T-P-X₁₈-G-G-;
-L/I-X-V/L-Xₐ-L-X₁₉-F-X₃-G-X₇-T-P-;
-L-X-K/R-X₁₇-F-X₃-G-X₇-T-P-;
-L-X-K/R-L/M-X₁₆-F-X₃-G-X₇-T-P-;
-L-X-K/R-L/M-M-X₁₅-F-X₃-G-X₇-T-P-;
-L-X-K/R-L/M-M-X₁₅-F-X₃-G-X₇-T-P-X₁₈-G-G-;
-L-X-K/R-L/M-M-X₁₅-F-X₃-G-X₇-T-P-X₂-L-;
-L-X-K/R-L/M-M-X₁₅-F-X₃-G-X₇-T-P-X₂-L-D/E-X₂-D/E-;
-L-X-K/R-L/M-M-X₁₅-F-X₃-G-X₇-T-P-X₂-L-D/E-X₂-D/E-
X₃-I-D/E-;
-L-X-K/R-L/M-M-X₁₅-F-X₃-G-X₇-T-P-X₂-L-D/E-X₂-D/E-
X₃-I-D/E-X₆-G-G-;
-L/I-X-V/L-Xₐ-L-X-K/R-L/M-M-X₁₅-F-X₃-G-X₇-T-P-X₂-
L-D/E-X₂-D/E-X₃-I-D/E-X₆-G-G-;
-L-K-V-K-X_{b}-L-X₁₉-F-X₃-G-X₇-T-P-;
-L-K-V-K-X_{b}-L-X-K-X-M-X₁₅-F-X₃-G-X₇-T-P-;
-L-K-V-K-X_{b}-L-X-K-X-M-X₂-Y-X₁₂-F-X₃-G-X₇-T-P-;
-L-K-V-K-X_{b}-L-X-K-X-M-X₂-Y-X₁₂-F-X₃-G-X₇-T-P-X₂-
L-;
-L-K-V-K-X_{b}-L-X-K-X-M-X₂-Y-X₁₂-F-X₃-G-X₇-T-P-X₂-L-
X₁₅-G-G-;
-L-K-V-K-X_{b}-L-X-K-X-M-X₂-Y-X₁₂-F-X₃-G-X₇-T-P-X₂-L-
X₇-I-;
-L-K-V-K-X_{b}-L-X-K-X-M-X₂-Y-X₁₂-F-X₃-G-X₇-T-P-X₂-L-
X₅-X-I-;
-L-K-V-K-X_{b}-L-X-K-X-M-X₂-Y-X₁₂-F-X₃-G-X₇-T-P-X₂-L-
X₇-I-X₇-G-G-;
-L-K-V-K-X-Q-X_{c}-L-X-K-X-M-X₂-Y-X₁₂-F-X₃-G-X₇-T-P-
X₂-L-X₇-I-X₇-G-G-;
-L-K-V-K-X_{b}-L-L/K-K-L/M-M-X₂-Y-X₁₂-F-X₃-G-X₇-T-P-
X₂-L-X₇-I-X₇-G-G-;
-L-K-V-K-X-Q-X_{c}-L-L/K-K-L/M-M-X₂-Y-X₁₂-F-X₃-G-X₇-
T-P-X₂-L-X₇-I-X₇-G-G-;
-L-X-K/R-L-X₁₆-F-X₃-G-X₇-T-P-;
-L-X-K/R-L-M-X₁₅-F-X₃-G-X₇-T-P-;
-L-X-K/R-L-M-X₅-R/K-Q/R-X₈-F-X₃-G-X₇-T-P-;
-L-X-K/R-L-M-X₅-R-Q-X₈-F-X₃-G-X₇-T-P-;
-L-X-K/R-L-M-X₅-R-Q-X₈-F-X₃-G-X₇-T-P-X₂-L-X-M-;
-L-X-K/R-L-M-X₅-R-Q-X₃-M-X₄-F-L-X₂-G-X₇-T-P-X₂-L-
X-M-;
-L-X-K/R-L-M-X₅-R-Q-X₈-F-L-X₂-G-X₇-T-P-X₂-L-X-M-E-
X₄-I-X₇-G-G-:
-L-X-K/R-L-M-X₅-R-Q-X₈-F-L-X₂-G-X-R-X₅-T-P-X₂-L-X-
M-E-X₄-I-X₇-G-G-:
wherein
the amino acid consensus sequences are given in N-terminal to C-terminal direction;
a is an integer of 17 or 18;
b is an integer of 16 or 17;
c is an integer of 14 or 15;
each letter stands for an amino acid residue;
X stands for any amino acid residue;
letters other than X stand for amino acid residues in the standard one-letter code;
a numerical subscript to a letter indicates that the amino acid residue defined by said letter is present contiguously and connected by peptide bonds as many times as indicated by the numerical value of the subscript;
“-” stands for a peptide bond connecting adjacent amino acid residues; and
“/” indicates that the amino acid position defined by two consecutive “-” can be occupied by any of the amino acid residues defined by letters separated by “/”.

12. The process according to claim 1, wherein said protease or a polypeptide comprising said protease is isolated from vegetative tissue of said plant.

13. The process according to claim 1, wherein said plant or said plant cells belong to genus Nicotiana.

14. The process according to claim 1, wherein said coding sequence contains one or more introns, notably in a region coding for said SUMO protein or said derivative of a SUMO protein.

15. The process according to claim 1, wherein said fusion protein comprises an affinity tag for purifying said fusion protein or a fragment thereof by affinity purification.

16. The process according to claim 1, further comprising
(c) isolating and purifying said protease or said zymogen or a fusion protein comprising said protease from vegetative tissue of said plant or from said plant cells, optionally followed by
(d) generating said protease from said zymogen or from said fusion protein by proteolytic cleavage.

17. The process according to claim 1, wherein said protease is selected from trypsin and chymotrypsin.

18. Vector or nucleotide sequence comprising a coding sequence encoding a fusion protein, said fusion protein comprising, in N-terminal to C-terminal direction, an apoplast or plastid signal peptide, a SUMO protein or derivative of a SUMO protein, and a zymogen of a protease.

19. A transgenic plant or plant cells for expressing a protease or a fusion protein comprising said protease, said plant comprising a nucleotide sequence containing a promoter active in leaf tissue and, downstream of said promoter and operably linked thereto, a coding sequence encoding a fusion protein comprising in the following order in N-terminal to C-terminal direction a plastid or apoplast signal peptide, a SUMO protein or a derivative thereof, and a zymogen of said protease.


SEQUENCE LISTING (26 sequences)

SEQ ID NO: 1 - DNA, 15184 bp, Artificial Sequence
  expression vector
ccgggtaggggcccagcggccgctctagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacctgcatccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtcagatcaggatctcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggccaattcgtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcagatcctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgcctgatctggggaaccctgtggttggcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatctaagctagcttggaattggtaccacgcgtttcgacaaaatttagaacgaacttaattatgatctcaaatacattgatacatatctcatctagatctaggttatcattatgtaagaaagttttgacgaatatggcacgacaaaatggctagactcgatgtaattggtatctcaactcaacattatacttataccaaacattagttagacaaaatttaaacaactattttttatgtatgcaagagtcagcatatgtataattgattcagaatcgttttgacgagttcggatgtagtagtagccattatttaatgtacatactaatcgtgaatagtgaatatgatgaaacattgtatcttattgtataaatatccataaacacatcatgaaagacactttctttcacggtctgaattaattatgatacaattctaatagaaaacgaattaaattacgttgaattgtatgaaatctaattgaacaagccaaccacgacgacgactaacgttgcctggattgactcggtttaagttaaccactaaaaaaacggagctgtcatgtaacacgcggatcgagcaggtcacagtcatgaagccatcaaagcaaaagaactaatccaagggctgagatgattaattagtttaaaaattagttaacacgagggaaaaggctgtctgacagccaggtcacgttatctttacctgtggtcgaaatgattcgtgtctgtcgattttaattatttttttgaaaggccgaaaataaagttgtaagagataaacccgcctatataaattcatatattttcctctccgctttgaagttttagttttattgcaacaacaacaacaaattacaataacaacaaacaaaatacaaacaacaacaacatggcacaatttcaacaaacaattgacatgcaaactctccaagccgctgcgggacgcaacagcttggtgaatgatttggcatctcgtcgcgtttacgataatgcagtcgaggagctgaatgctcgttccagacgtcccaaggtaaaacaacatttcattcacatatatgaatacttttgtcattgagtacgaagaagacacttactacttgttgatgaaagtttccgcctttatacttatctatatcattttcatcatttcaaactagtatgaaattaggtgatgtttatatgatatcatggaacattaatctatagggaaactgttttgagttagttttgtataatatttttccctgtttgatgttaggttcatttctccaaggcagtgtctacggaacagacactgattgcaacaaacgcatatccggagttcgagatttcctttactcatacgcaatccgctgtgcactccttggccggaggccttcggtcacttgagttggagtatctcatgatgcaagttccgttcggctctctgacctacgacatcggcggaaacttctccgcgcacctcttcaaaggtaattttctttctctactcaattttctccaagatccaatatttgaagactgatctatagttaaaattaatctctactccattcttgttacctcaggtcgcgattacgttcactgctgcatgcctaatctggatgtacgtgacattgctcgccatgaaggacacaaggaagctatttacagttatgtgaatcgtttgaaaaggcagcagcgtcctgtgcctgaataccagagggcagctttcaacaactacgctgagaacccgcacttcgtccattgcgacaaacctttccaacagtgtgaattgacgacagcgtatggcactgacacctacgctgtagctctccatagcatttatgatatccctgttgaggagttcggttctgcgctactcaggaagaatgtgaaaacttgtttcgcggcctttcatttccatgagaatatgcttctagattgtgatacagtcacactcgatgagattggagctacgttccagaaatcaggtaacattccttagttacctttcttttctttttccatcataagtttatagattgtacatgctttgagatttttctttgcaaacaatctcaggtgataacctgagcttcttcttccataatgagagcactctcaattacacccacagcttcagcaacatcatcaagtacgtgtgcaagacgttcttccctgctagtcaacgcttcgtgtaccacaaggagttcctggtcactagagtcaacacttggtactgcaagttcacgagagtggatacgttcactctgttccgtggtgtgtaccacaacaatgtggattgcgaagagttttacaaggctatggacgatgcgtggcactacaaaaagacgttagcaatgcttaatgccgagaggaccatcttcaaggataacgctgcgttaaacttctggttcccgaaggtgctcttgaaattggaagtcttcttttgttgtctaaacctatcaatttctttgcggaaatttatttgaagctgtagagttaaaattgagtcttttaaacttttgtaggtgagagacatggttatcgtccctctctttgacgcttctatcacaactggtaggatgtctaggagagaggttatggtgaacaaggacttcgtctacacggtcctaaatcacatcaagacctatcaagctaaggcactgacgtacgcaaacgtgctgagcttcgtggagtctattaggtctagagtgataattaacggtgtcactgccaggtaagttgttacttatgattgttttcctctctgctacatgtattttgttgttcatttctgtaagatataagaattgagttttcctctgatgatattattaggtctgaatgggacacagacaaggcaattctaggtccattagcaatgacattcttcctgatcacgaagctgggtcatgtgcaagatgaaataatcctgaaaaagttccagaagttcgacagaaccaccaatgagctgatttggacaagtctctgcgatgccctgatgggggttattccctcggtcaaggagacgcttgtgcgcggtggttttgtgaaagtagcagaacaagccttagagatcaaggttagtatcatatgaagaaatacctagtttcagttgatgaatgctattttctgacctcagttgttctcttttgagaattatttcttttctaatttgcctgatttttctattaattcattaggttcccgagctatactgtaccttcgccgaccgattggtactacagtacaagaaggcggaggagttccaatcgtgtgatctttccaaacctctagaagagtcagagaagtactacaacgcattatccgagctatcagtgcttgagaatctcgactcttttgacttagaggcgtttaagactttatgtcagcagaagaatgtggacccggatatggcagcaaaggtaaatcctggtccacacttttacgataaaaacacaagattttaaactatgaactgatcaataatcattcctaaaagaccacacttttgttttgtttctaaagtaatttttactgttataacaggtggtcgtagcaatcatgaagtcagaattgacgttgcctttcaagaaacctacagaagaggaaatctcggagtcgctaaaaccaggagaggggtcgtgtgcagagcataaggaagtgttgagcttacaaaatgatgctccgttcccgtgtgtgaaaaatctagttgaaggttccgtgccggcgtatggaatgtgtcctaagggtggtggtttcgacaaattggatgtggacattgctgatttccatctcaagagtgtagatgcagttaaaaagggaactatgatgtctgcggtgtacacagggtctatcaaagttcaacaaatgaagaactacatagattacttaagtgcgtcgctggcagctacagtctcaaacctctgcaaggtaagaggtcaaaaggtttccgcaatgatccctctttttttgtttctctagtttcaagaatttgggtatatgactaacttctgagtgttccttgatgcatatttgtgatgagacaaatgtttgttctatgttttaggtgcttagagatgttcacggcgttgacccagagtcacaggagaaatctggagtgtgggatgttaggagaggacgttggttacttaaacctaatgcgaaaagtcacgcgtggggtgtggcagaagacgccaaccacaagttggttattgtgttactcaactgggatgacggaaagccggtttgtgatgagacatggttcagggtggcggtgtcaagcgattccttgatatattcggatatgggaaaacttaagacgctcacgtcttgcagtccaaatggtgagccaccggagcctaacgccaaagtaattttggtcgatggtgttcccggttgtggaaaaacgaaggagattatcgaaaaggtaagttctgcatttggttatgctccttgcattttaggtgttcgtcgctcttccatttccatgaatagctaagattttttttctctgcattcattcttcttgcctcagttctaactgtttgtggtatttttgttttaattattgctacaggtaaacttctctgaagacttgattttagtccctgggaaggaagcttctaagatgatcatccggagggccaaccaagctggtgtgataagagcggataaggacaatgttagaacggtggattccttcttgatgcatccttctagaagggtgtttaagaggttgtttatcgatgaaggactaatgctgcatacaggttgtgtaaatttcctactgctgctatctcaatgtgacgtcgcatatgtgtatggggacacaaagcaaattccgttcatttgcagagtcgcgaactttccgtatccagcgcattttgcaaaactcgtcgctgatgagaaggaagtcagaagagttacgctcaggtaaagcaactgtgttttaatcaatttcttgtcaggatatatggattataacttaatttttgagaaatctgtagtatttggcgtgaaatgagtttgctttttggtttctcccgtgttataggtgcccggctgatgttacgtatttccttaacaagaagtatgacggggcggtgatgtgtaccagcgcggtagagagatccgtgaaggcagaagtggtgagaggaaagggtgcattgaacccaataaccttaccgttggagggtaaaattttgaccttcacacaagctgacaagttcgagttactggagaagggttacaaggtaaagtttccaactttcctttaccatatcaaactaaagttcgaaactttttatttgatcaacttcaaggccacccgatctttctattcctgattaatttgtgatgaatccatattgacttttgatggttacgcaggatgtgaacactgtgcacgaggtgcaaggggagacgtacgagaagactgctattgtgcgcttgacatcaactccgttagagatcatatcgagtgcgtcacctcatgttttggtggcgctgacaagacacacaacgtgttgtaaatattacaccgttgtgttggacccgatggtgaatgtgatttcagaaatggagaagttgtccaatttccttcttgacatgtatagagttgaagcaggtctgtctttcctatttcatatgtttaatcctaggaatttgatcaattgattgtatgtatgtcgatcccaagactttcttgttcacttatatcttaactctctctttgctgtttcttgcaggtgtccaatagcaattacaaatcgatgcagtattcaggggacagaacttgtttgttcagacgcccaagtcaggagattggcgagatatgcaattttactatgacgctcttcttcccggaaacagtactattctcaatgaatttgatgctgttacgatgaatttgagggatatttccttaaacgtcaaagattgcagaatcgacttctccaaatccgtgcaacttcctaaagaacaacctattttcctcaagcctaaaataagaactgcggcagaaatgccgagaactgcaggtaaaatattggatgccagacgatattctttcttttgatttgtaactttttcctgtcaaggtcgataaattttattttttttggtaaaaggtcgataatttttttttggagccattatgtaattttcctaattaactgaaccaaaattatacaaaccaggtttgctggaaaatttggttgcaatgatcaaaagaaacatgaatgcgccggatttgacagggacaattgacattgaggatactgcatctctggtggttgaaaagttttgggattcgtatgttgacaaggaatttagtggaacgaacgaaatgaccatgacaagggagagcttctccaggtaaggacttctcatgaatattagtggcagattagtgttgttaaagtctttggttagataatcgatgcctcctaattgtccatgttttactggttttctacaattaaaggtggctttcgaaacaagagtcatctacagttggtcagttagcggactttaactttgtggatttgccggcagtagatgagtacaagcatatgatcaagagtcaaccaaagcaaaagttagacttgagtattcaagacgaatatcctgcattgcagacgatagtctaccattcgaaaaagatcaatgcgattttcggtccaatgttttcagaacttacgaggatgttactcgaaaggattgactcttcgaagtttctgttctacaccagaaagacacctgcacaaatagaggacttcttttctgacctagactcaacccaggcgatggaaattctggaactcgacatttcgaagtacgataagtcacaaaacgagttccattgtgctgtagagtacaagatctgggaaaagttaggaattgatgagtggctagctgaggtctggaaacaaggtgagttcctaagttccatttttttgtaatccttcaatgttattttaacttttcagatcaacatcaaaattaggttcaattttcatcaaccaaataatatttttcatgtatatataggtcacagaaaaacgaccttgaaagattatacggccggaatcaaaacatgtctttggtatcaaaggaaaagtggtgatgtgacaacctttattggtaataccatcatcattgccgcatgtttgagctcaatgatccccatggacaaagtgataaaggcagctttttgtggagacgatagcctgatttacattcctaaaggtttagacttgcctgatattcaggcgggcgcgaacctcatgtggaacttcgaggccaaactcttcaggaagaagtatggttacttctgtggtcgttatgttattcaccatgatagaggagccattgtgtattacgatccgcttaaactaatatctaagttaggttgtaaacatattagagatgttgttcacttagaagagttacgcgagtctttgtgtgatgtagctagtaacttaaataattgtgcgtatttttcacagttagatgaggccgttgccgaggttcataagaccgcggtaggcggttcgtttgctttttgtagtataattaagtatttgtcagataagagattgtttagagatttgttctttgtttgataatgtcgatagtctcgtacgaacctaaggtgagtgatttcctcaatctttcgaagaaggaagagatcttgccgaaggctctaacgaggttaaaaaccgtgtctattagtactaaagatattatatctgtcaaggagtcggagactttgtgtgatatagatttgttaatcaatgtgccattagataagtatagatatgtgggtatcctaggagccgtttttaccggagagtggctagtgccagacttcgttaaaggtggagtgacgataagtgtgatagataagcgtctggtgaactcaaaggagtgcgtgattggtacgtacagagccgcagccaagagtaagaggttccagttcaaattggttccaaattactttgtgtccaccgtggacgcaaagaggaagccgtggcaggtaaggatttttatgatatagtatgcttatgtattttgtactgaaagcatatcctgcttcattgggatattactgaaagcatttaactacatgtaaactcacttgatgatcaataaacttgattttgcaggttcatgttcgtatacaagacttgaagattgaggcgggttggcagccgttagctctggaagtagtttcagttgctatggtcaccaataacgttgtcatgaagggtttgagggaaaaggtcgtcgcaataaatgatccggacgtcgaaggtttcgaaggtaagccatcttcctgcttatttttataatgaacatagaaataggaagttgtgcagagaaactaattaacctgactcaaaatctaccctcataattgttgtttgatattggtcttgtattttgcaggtgtggttgacgaattcgtcgattcggttgcagcatttaaagcggttgacaactttaaaagaaggaaaaagaaggttgaagaaaagggtgtagtaagtaagtataagtacagaccggagaagtacgccggtcctgattcgtttaatttgaaagaagaaaacgtcttacaacattacaaacccgaatcagtaccagtatttcgataagaaacaagaaatggggaagcaaatggccgccctgtgtggctttctcctcgtggcgttgctctggctcacgcccgacgtcgcgcatggtatgtctgcaaaccaggaggaagacaagaagccaggagacggaggagctcacatcaatctcaaagtcaagggacaggtatctctctttctccttctcatccttgtgtgttcttgtgaatgtttgggtttctgatttcgtgtagctgcgattagggtttttcattctccaatttggtttaattttagggtttcgtactactagctcagtcttaatggtcttcttcctcttttgttttacaggtttaaagtatcctgctttatgtttacacgtttgcttgtttttgctaattgtttatcaaatttctgaaattatataagtcttgtttaggtgtaagttgttattgagcttttggttcctgttgttgtttgagggttttatagttttggaagggataagtttacttagttgttgaatgttctaaagactgtgaacgatgctgtctctgtactaagttgttatatatcttctgatgaagttgtagtctctgtactaagttttcaattagtttggctgatgtttgtgcttcaaattctcatagacgaagccttaaactgaatgtttgttgatatgtgtttaatttttcgactctttcaggttactaccaaaagagagccatgtgatttagttattgtttcatatgcggtctctaacgtttattgttccttctatatgtttgttctataggatggaaacgaggttttctttaggatcaagagaagcactcagctcaagaagctgatgaatgcttactgtgaccggcaatctgtggacatgaactccattgctttcttgtttgatgggcgtcgtcttcgtgctgagcaaactcccgatgaggtataacattcattctacatgctttatttcttaccttttgaagtttatagtttctgatactaataattcttgacactacagcttgacatggaggatggtgatgagatcgatgcgatgcttcatcagactggaggtttccccgtggacgatgatgacaagatcgtgggcggctacacctgtggggcaaatactgtcccctaccaagtgtccctgaactctggctaccacttctgcgggggctccctcatcaacagccagtgggtggtgtctgcggctcactgctacaagtccggaatccaagtgcgtctgggagaagacaacattaatgtcgttgagggcaatgagcaattcatcagcgcatccaagagcatcgtccatcccagctacaactcaaacaccttaaacaacgacatcatgctgattaaactgaaatcagctgccagtctcaacagccgagtagcctctatctctctgccaacatcctgtgcctctgctggcacccagtgtctcatctctggctggggcaacaccaaaagcagtggcaccagctaccctgatgtcctgaagtgtctgaaggctcccatcctatcagacagctcttgcaaaagtgcctacccaggccagatcaccagcaacatgttctgtgcgggctacctggagggcggaaaggactcctgccagggtgactccggtggccctgtggtctgcagtggaaagctccagggcattgtctcctggggctctggctgcgctcagaaaaacaagcctggtgtctacaccaaggtctgcaactacgtgagctggattaagcagaccatcgcctccaactaaagcttactagagcgtggtgcgcacgatagcgcatagtgtttttctctccacttgaatcgaagagatagacttacggtgtaaatccgtaggggtggcgtaaaccaaattacgcaatgttttgggttccatttaaatcgaaaccccttatttcctggatcacctgttaacgcacgtttgacgtgtattacagtgggaataagtaaaagtgagaggttcgaatcctccctaacc

SEQ ID NO: 2 - DNA, 78 bp, Artificial Sequence
  sequence coding for apoplast signal peptide
atggggaagcaaatggccgccctgtgtggctttctcctcgtggcgttgctctggctcacgcccgacgtcgcgcatggt

SEQ ID NO: 3 - DNA, 180 bp, Artificial Sequence
  sequence coding for plastid signal peptide
atggcttcttctatgctttcttctgctgctgttgttgctactcgtgctagtgctgctcaagctagtatggttgctccttttactggacttaagtctgctgcttcttttcctgttactagaaagcaaaacaaccttgatattacttctattgctagtaacggaggaagagtccaatgcgca

SEQ ID NO: 4 - protein, 243 aa, bos taurus
FIFLALLGAAVAFPVDDDDKIVGGYTCGANTVPYQVSLNSGYHFCGGSLINSQWVVSAAHCYKSGIQVRLGEDNINVVEGNEQFISASKSIVHPSYNSNTLNNDIMLIKLKSAASLNSRVASISLPTSCASAGTQCLISGWGNTKSSGTSYPDVLKCLKAPILSDSSCKSAYPGQITSNMFCAGYLEGGKDSCQGDSGGPVVCSGKLQGIVSWGSGCAQKNKPGVYTKVCNYVSWIKQTIASN

SEQ ID NO: 5 - DNA, 825 bp, Bos taurus
cttcatctttctggctctcttgggagccgctgttgctttccccgtggacgatgatgacaagatcgtgggcggctacacctgtggggcaaatactgtcccctaccaagtgtccctgaactctggctaccacttctgcgggggctccctcatcaacagccagtgggtggtgtctgcggctcactgctacaagtccggaatccaagtgcgtctgggagaagacaacattaatgtcgttgagggcaatgagcaattcatcagcgcatccaagagtatcgtccatcccagctacaactcaaacaccttaaacaacgacatcatgctgattaaactgaaatcagctgccagtctcaacagccgagtagcctctatctctctgccaacatcctgtgcctctgctggcacccagtgtctcatctctggctggggcaacaccaaaagcagtggcaccagctaccctgatgtcctgaagtgtctgaaggctcccatcctatcagacagctcttgcaaaagtgcctacccaggccagatcaccagcaacatgttctgtgcgggctacctggagggcggaaaggactcctgccagggtgactccggtggccctgtggtctgcagtggaaagctccagggcattgtctcctggggctctggctgcgctcagaaaaacaagcctggtgtctacaccaaggtctgcaactacgtgagctggattaagcagaccatcgcctccaactaaatagcttcatctcttcatgaccctctctgctagccagcttcaccttcctcccatcctgaacgcactacttaaataaaatcatttataaaacc

SEQ ID NO: 6 - DNA, 1060 bp, Artificial Sequence
  synthetic EPB-2 gene
ggtctcaaggtattcctatggaagataaggatcttgagtctgaagaggctctttgggatctttatgagaggtggcagtctgctcatagagtgagaaggcatcatgctgagaaacatagaagattcggtactttcaagtctaatgctcatttcattcattctcataataagaggggtgatcatccttacaggcttcatcttaatagattcggtgatatggatcaggctgagttcagggctactttcgttggtgatcttagaagggatactccttctaagcctccttctgtgcctggttttatgtacgctgctcttaatgtgtctgatcttcctccatctgttgattggaggcagaagggtgctgttactggagttaaggatcagggaaagtgtggttcttgctgggctttctctactgttgtttctgtggagggtatcaatgctattaggactggttctcttgtgtctctttctgagcaagagcttattgattgcgatactgctgataatgatggttgccagggtggtcttatggataatgctttcgagtacattaagaacaatggtggtcttattactgaggctgcttacccttatagagctgctaggggaacttgtaacgttgctagggctgctcagaattctcctgtggtggtgcatattgatggtcatcaggatgtgcctgctaattctgaagaggatttggctagggctgttgctaatcagcctgtttctgttgctgttgaggcttctggaaaggctttcatgttctactctgagggagtttttactggtgagtgcggtactgaacttgatcatggtgttgctgttgttggttacggtgttgctgaagatggaaaggcttactggactgtgaagaattcttggggaccttcttggggtgaacagggttacattagggtggagaaggattctggtgcttctggtggtctttgcggtattgcaatggaagctagttaccctgttaagacttactctaagcctaagcctactcctagaagagctttgggtgctagagagtctctttaagctttgagacc

SEQ ID NO: 7 - protein, 345 aa, Artificial Sequence
  expression porduct of synthetic EPB-2 gene
IPMEDKDLESEEALWDLYERWQSAHRVRRHHAEKHRRFGTFKSNAHFIHSHNKRGDHPYRLHLNRFGDMDQAEFRATFVGDLRRDTPSKPPSVPGFMYAALNVSDLPPSVDWRQKGAVTGVKDQGKCGSCWAFSTVVSVEGINAIRTGSLVSLSEQELIDCDTADNDGCQGGLMDNAFEYIKNNGGLITEAAYPYRAARGTCNVARAAQNSPVVVHIDGHQDVPANSEEDLARAVANQPVSVAVEASGKAFMFYSEGVFTGECGTELDHGVAVVGYGVAEDGKAYWTVKNSWGPSWGEQGYIRVEKDSGASGGLCGIAMEASYPVKTYSKPKPTPRRALGARESL

SEQ ID NO: 8 - DNA, 30 bp, Artificial Sequence
  PCR primer
ggtctcaaggtttccccgtggacgatgatg

SEQ ID NO: 9 - DNA, 30 bp, Artificial Sequence
  PCR primer
ggtctcaaggtatcgtgggcggctacacct

SEQ ID NO: 10 - DNA, 30 bp, Artificial Sequence
  PCR primer
ggtctcatccggacttgtagcagtgagccg

SEQ ID NO: 11 - DNA, 27 bp, Artificial Sequence
  PCR primer
ggtctcacggaatccaagtgcgtctgg

SEQ ID NO: 12 - DNA, 29 bp, Artificial Sequence
  PCR primer
ggtctcatgccactgcttttggtgttgcc

SEQ ID NO: 13 - DNA, 29 bp, Artificial Sequence
  PCR primer
ggtctcaggcaccagctaccctgatgtcc

SEQ ID NO: 14 - DNA, 26 bp, Artificial Sequence
  PCR primer
ggtctcactggcaggagtcctttccg

SEQ ID NO: 15 - DNA, 30 bp, Artificial Sequence
  PCR primer
ggtctcaccagggtgactccggtggccctg

SEQ ID NO: 16 - DNA, 33 bp, Artificial Sequence
  PCR primer
ggtctcaagctttagttggaggcgatggtctgc

SEQ ID NO: 17 - protein, 95 aa, Mus musculus
MADEKPKEGVKTENNDHINLKVAGQDGSVVQFKIKRHTPLSKLMKAYCERQGLSMRQIRFRFDGQPINETDTPAQLEMEDEDTIDVFQQQTGGVY

SEQ ID NO: 18 - protein, 82 aa, Mus musculus
MSEEKPKEGVKTENDHINLKVAGQDGSVVQFKIKRHTPLSKLMKAYCERQGLSMRQIRFRFDGQPINETDTPAQFLALTILL

SEQ ID NO: 19 - protein, 101 aa, Mus musculus
MSDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGGHSTV

SEQ ID NO: 20 - protein, 100 aa, Oryza sativa
MSAAGEEDKKPAGGEGGGAHINLKVKGQDGNEVFFRIKRSTQLKKLMNAYCDRQSVDMNAIAFLFDGRRLRGEQTPDELEMEDGDEIDAMLHQTGGCLPA

SEQ ID NO: 21 - protein, 101 aa, Saccharomyces cerevisiae
MSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGATY

SEQ ID NO: 22 - protein, 100 aa, Arabidopsis thaliana
MSANQEEDKKPGDGGAHINLKVKGQDGNEVFFRIKRSTQLKKLMNAYCDRQSVDMNSIAFLFDGRRLRAEQTPDELDMEDGDEIDAMLHQTGGSGGGATA

SEQ ID NO: 23 - protein, 103 aa, Arabidopsis thaliana
MSATPEEDKKPDQGAHINLKVKGQDGNEVFFRIKRSTQLKKLMNAYCDRQSVDFNSIAFLFDGRRLRAEQTPDELEMEDGDEIDAMLHQTGGGAKNGLKLFCF

SEQ ID NO: 24 - protein, 111 aa, Arabidopsis thaliana
MSNPQDDKPIDQEQEAHVILKVKSQDGDEVLFKNKKSAPLKKLMYVYCDRRGLKLDAFAFIFNGARIGGLETPDELDMEDGDVIDACRAMSGGLRANQRQWSYMLFDHNGL

SEQ ID NO: 25 - protein, 114 aa, Arabidopsis thaliana
MSTTSRVGSNEVKMEGQKRKVVSDPTHVTLKVKGQDEEDFRVFWVRRNAKLLKMMELYTKMRGIEWNTFRFLFDGSRIREYHTPDELERKDGDEIDAMLCQQSGFGPSSIKFRV

SEQ ID NO: 26 - protein, 108 aa, Arabidopsis thaliana
MVSSTDTISASFVSKKSRSPETSPHMKVTLKVKNQQGAEDLYKIGTHAHLKKLMSAYCTKRNLDYSSVRFVYNGREIKARQTPAQLHMEEEDEICMVMELGGGGPYTP